(19) United States
(12) Patent Application Publication
(10) Pub. No.: US 2023/0304005 A1
(43) Pub. Date: Sep. 28, 2023

(54) Micro-Vesicles Comprising Cargo Prodrug RNA and Methods of Using the Same

(71) Applicant: The Board of Trustees of the Leland Stanford Junior University, Stanford, CA (US)

(72) Inventors: Stanley N. Cohen, Stanford, CA (US); Yanan Feng, Stanford, CA (US); Weijing Xu, Stanford, CA (US); Ning Deng, Stanford, CA (US)

(21) Appl. No.: 18/021,913

(22) PCT Filed: Aug. 24, 2021
(86) PCT No.: PCT/US2021/047262

§ 371 Date: Feb. 17, 2023

Related U.S. Application Data
(60) Provisional application No. 63/071,018, filed on Aug. 27, 2020.

(51) Int. Cl. C07K 14/47; C12N 15/113; A61P 31/14

(52) U.S. Cl. CPC C12N 15/113; A61P 31/14; C07K 14/4702; C07K 2319/85

(57) ABSTRACT

Aspects of the invention include micro-vesicles comprising cargo RNA. In some instances, the micro-vesicles include: (1) a TSG101 associating protein stably associated with a ribonucleic-acid-binding protein (RNA-binding protein); and (2) at least one cargo RNA complex that includes an RNA bound non-covalently to the RNA-binding protein and a cargo prodrug RNA component. Also provided are methods of making and using the micro-vesicles, e.g., in the treatment of disease conditions.

### CROSS-REFERENCE TO RELATED APPLICATIONS

Pursuant to 35 U.S.C. § 119 (e), this application claims priority to the filing date of United States Provisional Pat. Application Serial No. 63/071,018, filed Aug. 27, 2020, the disclosure of which applications is incorporated herein by reference.

### INTRODUCTION

Coronaviruses are enveloped, positive-sense single-stranded RNA viruses. They have the largest genomes (26-32 kb) among known RNA viruses, and are phylogenetically divided into four genera (alpha, beta, gamma, delta), with betacoronaviruses further subdivided into four lineages (A, B, C, D). Coronaviruses infect a wide range of avian and mammalian species, including humans. Of the six known human coronaviruses, four of them (HCoV-OC43, HCoV-229E, HCoV-HKU1 and HCoV-NL63) circulate annually in humans and generally cause mild respiratory diseases, although severity can be greater in infants, elderly, and the immunocompromised. In contrast, the Middle East respiratory syndrome coronavirus (MERS-CoV) and the severe acute respiratory syndrome coronavirus (SARS-CoV), belonging to betacoronavirus lineages C and B, respectively, are highly pathogenic.

In 2019, a novel coronavirus (2019-nCoV/SARS-CoV-2) instigated a major outbreak of respiratory disease, which was originally centered on Hubei province, China. The disease is now a global pandemic which is showing little signs of abatement. Taxonomically, SARS-CoV-2 is a betacoronavirus, which is thought to be of lineage A or C (Jaimes et al., “Phylogenetic Analysis and Structural Modeling of SARS-CoV-2 Spike Protein Reveals an Evolutionary Distinct and Proteolytically Sensitive Activation Loop,” J. Mol. Biol. (May 1, 2020) 432(10): 3309-3325). COVID-19, the disease caused by SARS-CoV-2, may manifest with a number of clinical symptoms, including pneumonia, fever, dry cough, headache, and dyspnea. In some instances, the disease may progress to respiratory failure and death. Id.

While great effort and substantial resources have been and continue to be expended in the search for both effective treatments and vaccines for SARS-CoV-2, there is a continued need for innovative treatments for this pandemic.

### SUMMARY

Aspects of the invention include micro-vesicles comprising cargo prodrug RNA. In some instances, the micro-vesicles include: (1) a TSG101 associating protein stably associated with a ribonucleic-acid-binding protein (RNA-binding protein); and (2) at least one cargo prodrug RNA complex that includes an RNA bound non-covalently to the RNA-binding protein and a cargo prodrug RNA). Also provided are methods of making and using the micro-vesicles, e.g., in the treatment of disease conditions.

In some instances, aspects of embodiments of the invention include micro-vesicles comprising siRNA precursors that, when processed into siRNA in virus-infected cells, target virus RNA. In some instances, the micro-vesicles include: (1) a protein stably associated with both TSG101 and a ribonucleic-acid-binding protein (RNA-binding protein); and (2) at least one cargo complex that includes (a) an RNA (defined below as a “binding RNA) that is attached non-covalently to the RNA-binding protein and (b) a double-stranded ribonucleic acid (a “cargo-RNA”) that is capable, upon processing, of binding to a target, e.g., viral RNA, protein or metabolite target. Also provided are methods of making and using the micro-vesicles, e.g., in the treatment of viral conditions, including coronaviral conditions, e.g., COVID-19.

### BRIEF DESCRIPTION OF THE FIGURES

FIGS. 1A and 1B provide results showing that constructs of TAR-myc-shSupt4 and TAR-myc-shRdRP are efficient to knock down target gene RNA abundance (it is noted that the “myc” domain is a linker).

FIGS. 2A and 2B provide results showing that the interaction of TAT-TAR enables ARRDC1-mediated shRNA packaging in ARMMs.

FIGS. 3A and 3B show that elevated amounts of shRNA is present in ARMMs produced from 293 Dicer deficient cells as compared with 293 cells that contain a normal Dicer gene.

FIGS. 4A to 4C show the protection of ARMMs producing cells from the toxicity effect of Supt4 siRNA by using 293 dicer deficient cargo-producing cells.

### DEFINITIONS

The term “micro-vesicle” is used in its conventional sense and refers to extracellular vesicles (EV) that are released from the cell, where the micro-vesicles are delimited by a phospholipid bilayer. Micro-vesicles can vary in size, ranging in some instances from 30 to 1000 nm in diameter.

The term “ARMM,” as used herein, refers to a micro-vesicle comprising an ARRDC1 protein or variant thereof, and/or TSG101 protein or variant thereof. In some embodiments, an ARMM is shed from a cell, and comprises a molecule, for example, a nucleic acid, protein, or small molecule, present in the cytoplasm of the shedding cell or associated with the plasma membrane of the shedding cell. In some embodiments, the ARMM is shed from a transgenic cell comprising a recombinant expression construct that includes a transgene, and the ARMM comprises a gene product, for example, a transcript and/or a protein (e.g., an ARRDC1-Tat fusion protein and a TAR-cargo RNA) encoded by the expression construct.

The term “binding RNA”, as used herein, refers to a ribonucleic acid (RNA) that binds to an RNA binding protein, for example, any of the RNA binding proteins known in the art and/or provided herein. A binding RNA may be viewed as a ligand for an RNA binding protein, and may be referred to herein as an RNA ligand for that RNA binding protein. In some embodiments, a binding RNA (i.e., the RNA ligand) is an RNA that specifically binds to an RNA binding protein. A binding RNA that “specifically binds” to an RNA binding protein, is defined as an RNA that binds to such protein with greater affinity, avidity, more readily, and/or with greater duration than it binds to other proteins. In some embodiments, the binding RNA is a naturally-occurring RNA, or non-naturally-occurring variant thereof, that binds to a specific RNA binding protein. For example, the binding RNA may be a TAR element, a Rev response element, an MS2 RNA, or any variant thereof that specifically binds an RNA binding protein. In some embodiments, the binding RNA may be a trans-activating response element (TAR element), or variant thereof, which is an RNA stem-loop structure that is found at the 5′ ends of nascent HIV-1 transcripts and specifically binds to the trans-activator of transcription (Tat) protein. In some embodiments, the binding RNA is a Rev response element (RRE), or variant thereof, that specifically binds to the accessory protein Rev (e.g., Rev from HIV-1). In some embodiments, the binding RNA is an MS2 RNA that specifically binds to a MS2 phage coat protein. Binding RNAs of the present disclosure may be designed to specifically bind a protein (e.g., an RNA binding protein fused to ARRDC1) in order to facilitate loading of the binding RNA (e.g., a binding RNA fused to a cargo RNA) into a micro-vesicle, such as an ARMM.

The term “aptamer”, as used herein, refers to nucleic acids that bind to a specific target molecule, e.g., an RNA binding protein. In some embodiments, nucleic acid (e.g., DNA or RNA) aptamers are engineered through repeated rounds of in vitro selection or equivalently, SELEX (systematic evolution of ligands by exponential enrichment) methodology to bind to various molecular targets, for example, proteins, small molecules, macromolecules, metabolites, carbohydrates, metals, nucleic acids, cells, tissues, and organisms. Methods for engineering aptamers to bind to various molecular targets, such as proteins, are known in the art and include those described in U.S. Pat. Nos. 6,376,19; and 9,061,043; Shui B., et al., “RNA aptamers that functionally interact with green fluorescent protein and its derivatives.” Nucleic Acids Res., Mar; 40(5): e39 (2012); Trujillo U. H., et al., “DNA and RNA aptamers: from tools for basic research towards therapeutic applications”. Comb Chem High Throughput Screen 9 (8): 619-32 (2006); Srisawat C, et al., “Streptavidin aptamers: Affinity tags for the study of RNAs and ribonucleoproteins.” RNA, 7:632-641 (2001); and Tuerk and Gold, “Systematic evolution of ligands by exponential enrichment: RNA ligands to bacteriophage T4 DNA polymerase.” Science. 1990; the entire contents of each of which are hereby incorporated by reference in their entirety.

The term “RNA binding protein”, as used herein refers to a polypeptide molecule that binds to a binding RNA, e.g., as described above. In some embodiments, an RNA binding protein is a protein that specifically binds to a binding RNA. An RNA binding protein that “specifically binds” to a binding RNA, binds to the binding RNA with greater affinity, avidity, more readily, and/or with greater duration than it binds to another RNA, such as a control RNA (e.g., an RNA having a random nucleic acid sequence) or an RNA that weakly binds to the RNA binding protein. In some embodiments, the RNA binding protein is a naturally-occurring protein, or non-naturally-occurring variant thereof, that binds to a specific RNA. For example, in some embodiments, the RNA binding protein may be a trans-activator of transcription (Tat) protein that specifically binds a trans-activating response element (TAR element). In some embodiments, the RNA binding protein is a regulator of virion expression (Rev) protein (e.g., Rev from HIV-1) or variant thereof, that specifically binds to a Rev response element (RRE). In some embodiments, the RNA binding protein is a coat protein of an MS2 bacteriophage that specifically binds to an MS2 RNA. The RNA binding proteins useful in the present disclosure (e.g., a binding protein fused to ARRDC1) may be designed to specifically bind a binding RNA (e.g., a binding RNA fused to a cargo RNA) in order to facilitate loading of the binding RNA into an ARMM.

The term “cargo RNA”, as used herein, refers to a ribonucleic acid that may be incorporated into a micro-vesicle, such as an ARMM, for example, into the liquid phase of the micro-vesicle (e.g., by associating the cargo RNA with a binding RNA that specifically binds to an RNA binding protein fused to an ARRDC1 protein). The term “cargo RNA to be delivered” refers to any RNA that can be delivered via its association with or inclusion in a micro-vesicle to a subject, organ, tissue, or cell. In some embodiments, the cargo RNA is to be delivered to a targeted cell in vitro, in vivo, or ex vivo. In some instances, the cargo RNA is an inactive precursor of an RNA having activity that is desired in the target cell for which the micro-vesicle is configured for use. In other words, the cargo RNA is a prodrug cargo RNA, in that it is does not exhibit activity in the cell in which the micro-vesicle is produced, but may be processed to exhibit in the target cell for which the micro-vesicle is configured for use, as reviewed in greater detail below. For example, the cargo RNA to be delivered in some embodiments is an RNA that, upon processing in the target cell to produce siRNA, inhibits the expression of one or more genes in a cell. In yet another example, the prodrug cargo RNA may be an RNA incapable of replication in the micro-vesicle producing cell but capable of binding products made by an infectious viral pathogen in the targeted cell, e.g., where the cargo prodrug RNA contains some but not all of the RNA sequence of an infectious viral pathogen being treated and the cargo prodrug RNA or the RNA that results from processing is used as a “decoy” in the target cell to bind a viral RNA polymerase or another gene product made by the pathogenic virus infecting the targeted cell. In some embodiments, a cargo RNA to be delivered is a therapeutic agent. As used herein, the term “therapeutic agent” refers to any agent that, when administered to a subject, has a beneficial effect. In certain embodiments, the cargo RNA is associated with a binding RNA, either covalently or non-covalently (e.g., via nucleotide base pairing) to facilitate loading of the cargo RNA into a micro-vesicle, such as an ARMM.

The term “linker,” as used herein, refers to a chemical moiety linking two molecules or moieties, e.g., an ARRDC1 protein and a Tat protein, or a WW domain and a Tat protein. The linker may be positioned between, or flanked by, two groups, molecules, or other moieties and connected to each one via a covalent bond, thus connecting the two. In some embodiments, the linker includes an amino acid or a plurality of amino acids (e.g., a peptide or protein). In some embodiments, the linker comprises a nucleotide (e.g., DNA or RNA) or a plurality of nucleotides (e.g., a nucleic acid). In some embodiments, the linker is an organic molecule, group, polymer, or other chemical moiety. In some embodiments, the linker is a cleavable linker, e.g., the linker comprises a bond that can be cleaved upon exposure to, for example, UV light or a hydrolytic enzyme, such as a lysosomal protease. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids). In other embodiments, the linker is a chemical bond (e.g., a covalent bond).

As used herein, the term “animal” refers to any member of the animal kingdom. In some embodiments, the term “animal” refers to a human of either sex at any stage of development. In some embodiments, the term “animal” refers to a non-human animal at any stage of development. In certain embodiments, the non-human animal is a mammal (e.g., a rodent, a mouse, a rat, a rabbit, a monkey, a dog, a cat, a sheep, cattle, a primate, or a pig). Animals include, but are not limited to, mammals, birds, reptiles, amphibians, fish, and worms. In some embodiments, the animal is a transgenic animal, genetically-engineered animal, or a clone. In some embodiments, the animal is a transgenic non-human animal, genetically-engineered non-human animal, or a non-human clone.

As used herein, the term “associated with”, when used with respect to two or more entities, for example, with chemical moieties, molecules, and/or ARMMs, means that the entities are physically connected with one another, either directly or via one or more additional moieties that serve as a linker, to form a structure.

A micro-vesicle, e.g., an ARMM, is typically associated with an agent, for example, a nucleic acid, protein, or small molecule, by a mechanism that involves a covalent (e.g., via an amide bond) or non-covalent association (e.g., between ARRDC1 and a WW domain, or between a Tat protein and a TAR element). In certain embodiments, the agent to be delivered (e.g., a cargo RNA) is covalently bound to a molecule (e.g., a TAR element) that associates non-covalently with a protein or RNA encoded by a construct that is part of the ARMM, for example, a Tat protein, or variant thereof, that is fused to a TSG1010 associated protein, e.g., an ARRCD1 protein, or variant thereof. In some embodiments, the agent to be delivered (e.g., a cargo RNA) is covalently bound to a molecule (e.g., a TAR element) that associates non-covalently with a Tat protein, or variant thereof, that is fused to a WW domain, where the WW domain non-covalently associates with ARRDC1 in an ARMM. In some embodiments, the association is via a linker, for example, a cleavable linker. In some embodiments, an entity (e.g., a cargo RNA) is associated with an ARMM by inclusion in the ARMM, for example, by encapsulation of an entity (e.g., a cargo RNA) within the ARMM. For example, in some embodiments, an agent (e.g., a cargo RNA) present in the cytoplasm of an ARMM -producing cell is associated with an ARMM by encapsulation of the cytoplasm with the agent in the ARMM upon ARMM budding. Similarly, a membrane protein or other molecule associated with the cell membrane of an ARMM producing cell may be associated with an ARMM produced by the cell by inclusion into the ARMM’s membrane upon budding.

As used herein, the phrase “biologically active” refers to a characteristic of any substance that has activity in a cell, organ, tissue, and/or subject. For instance, a substance that, when administered to an organism, has a biological effect on that organism, is considered to be biologically active. As one example, a cargo RNA may be considered biologically active if it decreases the expression of a gene product when administered to a subject or cell.

As used herein, the term “conserved” refers to nucleotides or amino acid residues of a polynucleotide sequence or amino acid sequence, respectively, that are those that occur unaltered in the same position of two or more related sequences being compared. Nucleotides or amino acids that are relatively conserved are those that are conserved amongst more related sequences than are nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95% identical, about 98% identical, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another.

The term “engineered,” as used herein refers to a protein, nucleic acid, complex, substance, or entity that has been designed, produced, prepared, synthesized, and/or manufactured by a human. In some embodiments, an engineered protein or nucleic acid is a protein or nucleic acid that has been designed to meet particular requirements or to have particular design features. For example, a cargo RNA may be engineered to associate with a TSG101 protein, e.g., ARRDC1, by fusing a Tat protein to the TSG101 protein and by fusing the cargo RNA to a TAR element (producing a cargo RNA complex) to facilitate loading of the cargo RNA into an ARMM. In another example, a cargo RNA may be engineered to associate with a TSG101 protein, e.g., ARRDC1, by fusing one or more WW domains to a Tat protein and fusing the cargo RNA to a TAR element to facilitate loading of the cargo RNA into micro-vesicle, e.g., an ARMM.

As used herein, a “fusion protein” includes a first protein moiety, e.g., an ARRCD1 protein or variant thereof, associated covalently with a second protein moiety, for example, a Tat protein. In certain embodiments, the fusion protein is encoded by a single fusion gene.

As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that the definition of gene includes references to nucleic acids that do not encode proteins but rather encode functional RNA molecules, such as gRNAs, RNAi agents, ribozymes, tRNAs, etc. For the purpose of clarity, it should be noted that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein-coding expression units but rather to clarify that, in most cases, the term as used herein refers to a protein-coding nucleic acid.

As used herein, the term “gene product” or “expression product” generally refers to an RNA transcribed from the gene (pre-and/or post-processing) or a polypeptide (pre-and/or post-modification) encoded by an RNA transcribed from the gene.

As used herein, the term “homology” refers to the overall relatedness between nucleic acids (e.g., DNA molecules and/or RNA molecules) or polypeptides. In some embodiments, nucleic acids or proteins are considered to be “homologous” to one another if their sequences are at least 50%, at least 55%, at least 60%, at least 65%, at least 70%, at least 75%, at least 80%, at least 85%, at least 90%, at least 95%, or at least 99% identical. In some embodiments, nucleic acids or proteins are considered to be “homologous” to one another if their sequences are at least 50%, at least 55%, at least 60%, at least 65%, at least 70%, at least 75%, at least 80%, at least 85%, at least 90%, at least 95%, or at least 99% similar. The term “homologous” necessarily refers to a comparison between at least two sequences (nucleotide sequences or amino acid sequences). In accordance with the invention, two nucleotide sequences are considered to be homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for sequences to be considered homologous. For nucleotide sequences less than 60 nucleotides in length, homology is determined by the ability to encode a stretch of at least 4-5 uniquely specified amino acids. In accordance with the invention, two protein sequences are considered to be homologous if the proteins are at least about 50% identical, at least about 60% identical, at least about 70% identical, at least about 80% identical, or at least about 90% identical for at least one stretch of at least about 20 amino acids.

As used herein, the term “identity” refers to the overall relatedness between nucleic acids or proteins (e.g., DNA molecules, RNA molecules, and/or polypeptides). Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol, 215, 403 (1990)).

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe).

As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated substances are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

As used herein, the term “nucleic acid,” in its broadest sense, refers to a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleotides. As used herein, the terms “oligonucleotide” and “polynucleotide” can be used interchangeably to refer to a polymer of nucleotides (e.g., a string of at least two nucleotides). In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleoside(s) (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases( e.g., methylated bases); intercalated bases; modified sugars(e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery.

As used herein, the term “protein” refers to a string of at least two amino acids linked to one another by one or more peptide bonds. Proteins may include moieties other than amino acids (e.g., may be glycoproteins) and/or may be otherwise processed or modified. Those of ordinary skill in the art will appreciate that a “protein” can be a complete protein chain as produced by a cell (with or without a signal sequence) or can be a functional portion thereof. Those of ordinary skill will further appreciate that a protein can sometimes include more than one protein chain, for example linked by one or more disulfide bonds or associated by other means. Proteins may contain L-amino acids, D-amino acids, or both and may contain any of a variety of amino acid modifications or analogs known in the art. Useful modifications include, e.g., addition of a chemical entity such as a carbohydrate group, a phosphate group, a farnesyl group, an isofarnesyl group, a fatty acid group, an amide group, a terminal acetyl group, a linker for conjugation, functionalization, or other modification (e.g., alpha amidation), etc. In certain embodiments, the modifications of the protein lead to a more stable protein (e.g., greater half-life in vivo). These modifications may include cyclization of the protein, the incorporation of D-amino acids, etc. None of the modifications should substantially interfere with the desired biological activity of the protein. In certain embodiments, the modifications of the protein lead to a more biologically active protein. In some embodiments, proteins may comprise natural amino acids, non-natural amino acids, synthetic amino acids, amino acid analogs, and combinations thereof.

As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals, such as mice, rats, rabbits, non-human primates, and humans) and/or plants. In some embodiments, the subject is a patient having or suspected of having a disease or disorder. In other embodiments, the subject is a healthy volunteer.

As used herein, the term “therapeutically effective amount” means an amount of an agent to be delivered (e.g., nucleic acid, protein, drug, therapeutic agent, diagnostic agent, prophylactic agent, RNA, ARMM, or ARMM comprising a cargo RNA) that is sufficient, when administered to a subject suffering from or susceptible to a disease, disorder, and/or condition, to treat, improve symptoms of, diagnose, prevent, and/or delay the onset of the disease, disorder, and/or condition.

As used herein, the term “transcription factor” refers to a protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules. Examples of transcription factors include, but are not limited to, Sp1, NF1, SUPT4H, SUPT5H, CCAAT, GATA, HNF, PIT-1, MyoD, Myf5, Hox, Winged Helix, SREBP, p53, CREB, AP-1, Mef2, STAT, R-SMAD, NF-_{K}B, Notch, TUBBY, and NFAT.

As used herein, the term “treating” refers to partially or completely preventing, and/or reducing the incidence of one or more symptoms or features of a particular disease or condition. For example, “treating” a respirator disease may refer to inhibiting enhancing survival of the subject, diminishing one or more symptoms of the subject, suffering thereof. Treatment may be administered to a subject who does not exhibit signs or symptoms of a disease, disorder, and/or condition and/or to a subject who exhibits only early signs or symptoms of a disease, or condition for the purpose of decreasing the risk of developing more severe effects associated with the disease, disorder, or condition.

As used herein, “vector” refers to a nucleic acid molecule which can transport another nucleic acid to which it has been linked. In some embodiment, vectors can achieve extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

The term “WW domain” as used herein, refers to a protein domain having two basic residues at the C-terminus that mediates protein-protein interactions with short proline-rich or proline-containing motifs. It should be appreciated that the two basic residues (e.g., H, R, and K) of the WW domain are not required to be at the absolute C-terminal end of the WW protein domain. Rather, the two basic residues may be at a C-terminal portion of the WW protein domain (e.g., the C-terminal half of the WW protein domain). In some embodiments, the WW domain contains at least 1, 2, 3, 4, 5, 6, 7, 8, 9, or 10 W residues. In some embodiments, the WW domain contains at least two W residues. In some embodiments, the at least two W residues are spaced apart by from 15-25 amino acids. In some embodiments, the at least two W residues are spaced apart by from 19-23 amino acids. In some embodiments, the at least two W residues are spaced apart by from 20-22 amino acids. The WW domain possessing the two basic C-terminal amino acid residues may have the ability to associate with short proline-rich or proline-containing motifs (e.g., a PPXY motif). WW domains bind a variety of distinct peptide ligands including motifs with core proline-rich sequences, such as PPXY, which is found in AARDC1. A WW domain may be a 30-40 amino acid protein interaction domain with two signature tryptophan residues spaced by 20-22 amino acids. The three-dimensional structure of WW domains shows that they generally fold into a three-stranded, antiparallel β sheet with two ligand-binding grooves. WW domains are further described in PCT Application Serial No. PCT/US2017/054912 published as WO/2018/067546; the disclosure of which is herein incorporated by reference.

### DETAILED DESCRIPTION

Aspects of the invention include micro-vesicles comprising cargo prodrug RNA, e.g., viral RNA targeting siRNA precursors. In some instances, the micro-vesicles include: (1) a TSG101 associating protein stably associated with a ribonucleic-acid-binding protein (RNA-binding protein); and (2) at least one cargo RNA complex that includes an RNA bound non-covalently to the RNA-binding protein and a cargo prodrug RNA (e.g., double-stranded ribonucleic acid), e.g., that is capable of being processed into an RNA that has a desired activity, e.g., an RNA that binds to a target, such as a viral RNA, metabolite or protein target. Also provided are methods of making and using the micro-vesicles, e.g., in the treatment of disease conditions, such as viral conditions, including coronaviral conditions, e.g., COVID-19.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. §112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. §112 are to be accorded full statutory equivalents under 35 U.S.C. §112.

### Compositions

As summarized above, aspects of the invention include micro-vesicles that comprise a cargo that includes a prodrug RNA, such as viral RNA targeting siRNA precursors. In some instances, micro-vesicles of embodiments of the invention include a TSG101 protein, a TSG101 associating protein, and one or more prodrug RNA complexes. Each of these components is now reviewed in greater detail.

### TSG101 Protein

Micro-vesicles of embodiments of the invention include a TSG101 protein. By TSG101 protein is meant a TSG101 protein or active variant thereof (for ease of description, going forward the phrase “TSG101 protein” refers to a TSG101 protein or an active variant thereof). Tumor susceptibility gene 101, also referred to herein as TSG101, is a protein encoded by this gene and belonging to a group of apparently inactive homologs of ubiquitin-conjugating enzymes. The protein contains a coiled-coil domain that interacts with stathmin, a cytosolic phosphoprotein implicated in tumorigenesis. TSG101 is a protein that comprises a UEV domain and interacts with

ARRDC1, among other TSG101 associating proteins. Variants of TSG101 may also be present, such as fragments of TSG101 and/or TSG101 proteins that have a degree of identity (e.g., 60%, 70%, 80%, 85%, 90%, 95%, 98%, or 99% identity) to a TSG101 protein and are capable if interacting with ARRDC1. Accordingly, a TSG101 protein may be a protein that comprises a UEV domain, and interacts with a TSG101 associating protein, such as ARRDC. In some embodiments, the TSG101 protein is at least 60%, 65%, 70%, 75%, 80%, 85%, 90%, 95%, 98%, or 99% identical to the amino acid sequence of any one of SEQ ID NOs: 1 to 3, comprises a UEV domain, and interacts with ARRDC1. In some embodiments, the TSG101 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, or at least 390, identical contiguous amino acids of any one of SEQ ID NOs: 1 -3, comprises a UEV domain, and interacts with ARRDC1. In some embodiments, the TSG101 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 1-3 and comprises a UEV domain. In some embodiments, the TSG101 protein comprises any one of the amino acid sequences set forth in SEQ ID NOs: 1-3. Exemplary, non-limiting

TSG101 protein sequences are provided herein, and additional, suitable TSG101 protein sequences, isoforms, and variants according to aspects of this invention are known in the art. It will be appreciated by those of skill in the art that this invention is not limited in this respect. Exemplary TSG101 sequences include the following:

[>gi|5454140|ref|NP_006283.1| tumor susceptibility gene 101 protein [Homo sapiens]

1 MAVSESQLKK MVSKYKYRDL TVRETVNVIT LYKDLKPVLD SYVFNDGSSR ELMNLTGTIP
61 VPYRGNTYNI PICLWLLDTY PYNPPICFVK PTSSMTIKTG KHVDANGKIY LPYLHEWKHP
121 QSDLLGLIQV MIVVFGDEPP VFSRPISASY PPYQATGPPN TSYMPGMPGG ISPYPSGYPP
181 NPSGYPGCPY PPGGPYPATT SSQYPSQPPV TTVGPSRDGT ISEDTIRASL ISAVSDKLRW
241 RMKEEMDRAQ AELNALKRTE EDLKKGHQKL EEMVTRLDQE VAEVDKNIEL LKKKDEELSS
301 ALEKMENQSE NNDIDEVIIP TAPLYKQILN LYAEENAIED TIFYLGEALR RGVIDLDVFL
361 KHVRLLSRKQ FQLRALMQKA RKTAGLSDLY (SEQ ID NO:01)

>gi|11230780|ref|NP_068684.1| tumor susceptibility gene 101 protein [Mus musculus]

1 MAVSESQLKK MMSKYKYRDL TVRQTVNVIA MYKDLKPVLD SYVFNDGSSR ELVNLTGTIP
61 VRYRGNIYNI PICLWLLDTY PYNPPICFVK PTSSMTIKTG KHVDANGKIY LPYLHDWKHP
121 RSELLELIQI MIVIFGEEPP VFSRPTVSAS YPPYTATGPP NTSYMPGMPS GISAYPSGYP
181 PNPSGYPGCP YPPAGPYPAT TSSQYPSQPP VTTVGPSRDG TISEDTIRAS LISAVSDKLR
241 WRMKEEMDGA QAELNALKRT EEDLKKGHQK LEEMVTRLDQ EVAEVDKNIE LLKKKDEELS
301 SALEKMENQS ENNDIDEVII PTAPLYKQIL NLYAEENAIE DTIFYLGEAL RRGVIDLDVF
361 LKHVRLLSRK QFQLRALMQK ARKTAGLSDL Y (SEQ ID NO:02)

>gi|48374087|ref|NP_853659.2| tumor susceptibility gene 101 protein [Rattus norvegicus]

1 MAVSESQLKK MMSKYKYRDL TVRQTVNVIA MYKDLKPVLD SYVFNDGSSR ELVNLTGTIP
61 VRYRGNIYNI PICLWLLDTY PYNPPICFVK PTSSMTIKTG KHVDANGKIY LPYLHDWKHP
121 RSELLELIQI MIVIFGEEPP VFSRPTVSAS YPPYTAAGPP NTSYLPSMPS GISAYPSGYP
181 PNPSGYPGCP YPPAGPYPAT TSSQYPSQPP VTTAGPSRDG TISEDTIRAS LISAVSDKLR
241 WRMKEEMDGA QAELNALKRT EEDLKKGHQK LEEMVTRLDQ EVAEVDKNIE LLKKKDEELS
301 SALEKMENQS ENNDIDEVII PTAPLYKQIL NLYAEENAIE DTIFYLGEAL RRGVIDLDVF
361 LKHVRLLSRK QFQLRALMQK ARKTAGLSDL Y (SEQ ID NO:03)

The UEV domain in these sequences includes amino acids 1-145. The structure of UEV domains is known to those of skill in the art (see, e.g., Owen Pornillos et al., Structure and functional interactions of the Tsg101 UEV domain, EMBO J. 2002 May 15; 21(10): 2397-2406, the entire contents of which are incorporated herein by reference).

### TSG101 Associating Proteins and RNA Binding Proteins

In addition to the TSG101 protein, micro-vesicles of the invention also include a TSG101 associating protein. TSG101 associating proteins include, but are not limited to: ARRDC1, LRSAM1, RP300 and VPS28.

In some instances, the TSG101 associating protein is ARRDC1. ARRDC1 is a protein that includes a PSAP motif and a PPXY motif, also referred to herein as a PSAP and PPXY motif, respectively, in its C-terminus, and interacts with TSG101. The PSAP motif and the PPXY motif are not required to be at the absolute C-terminal end of the ARRDC1. Rather, they may be at a C-terminal portion of the ARRDC1 protein (e.g., the C-terminal half of the ARRDC1). The disclosure also contemplates variants of ARRDC1, such as fragments of ARRDC1 and/or ARRDC1 proteins that have a degree of identity (e.g., 60%, 70%, 80%, 85%, 90%, 95%, 98%, or 99% identity) to an ARRDC1 protein and are capable if interacting with TSG101 (for ease of description, going forward the phrase “ARRDC1 protein” refers to a ARRDC1 protein or an active variant thereof). Accordingly, an ARRDC1 protein may be a protein that comprises a PSAP motif and a PPXY motif and interacts with TSG101. In some embodiments, the ARRDC1 protein is at least 60%, 65%, 70%, 75%, 80%, 85%, 90%, 95%, 98%, or 99% identical to the amino acid sequence of any one of SEQ ID NOs: 4-6, comprises a PSAP motif and a PPXY motif, and interacts with TSG101. In some embodiments, the ARRDC1 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, or at least 430 identical contiguous amino acids of any one of SEQ ID NOs: 4-6, comprises a PSAP motif and a PPXY motif, and interacts with TSG101. In some embodiments, the ARRDC1 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 4-6 comprises a PSAP motif and a PPXY motif, and interacts with TSG101. In some embodiments, the ARRDC1 protein comprises any one of the amino acid sequences set forth in SEQ ID NOs: 4-6. Exemplary, non-limiting ARRDC1 protein sequences are provided herein, and additional, suitable ARRDC1 protein variants according to aspects of this invention are known in the art. It will be appreciated by those of skill in the art that this invention is not limited in this respect.

Exemplary ARRDC1 sequences include the following:

>gi|22748653|ref|NP_689498.1| arrestin domain-containing protein 1 [Homo sapiens]

1 MGRVQLFEIS LSHGRVVYSP GEPLAGTVRV RLGAPLPFRA IRVTCIGSCG VSNKANDTAW
61 VVEEGYFNSS LSLADKGSLP AGEHSFPFQF LLPATAPTSF EGPFGKIVHQ VRAAIHTPRF
121 SKDHKCSLVF YILSPLNLNS IPDIEQPNVA SATKKFSYKL VKTGSVVLTA STDLRGYVVG
181 QALQLHADVE NQSGKDTSPV VASLLQKVSY KAKRWIHDVR TIAEVEGAGV KAWRRAQWHE
241 QILVPALPQS ALPGCSLIHI DYYLQVSLKA PEATVTLPVF IGNIAVNHAP VSPRPGLGLP
301 PGAPPLVVPS APPQEEAEAE AAAGGPHFLD PVFLSTKSHS QRQPLLATLS SVPGAPEPCP
361 QDGSPASHPL HPPLCISTGA TVPYFAEGSG GPVPTTSTLI LPPEYSSWGY PYEAPPSYEQ
421 SCGGVEPSLT PES (SEQ ID NO:04)

>gi|244798004IreflNP_001155957.1| arrestin domain-containing protein 1 isoform a [Mus musculus]

MGRVQLFEIR LSQGRVVYGP GEPLAGTVHL RLGAPLPFRA IRVTCMGSCG VSTKANDGAW VVEESYFNSS
LSLADKGSLP AGEHNFPFQF LLPATAPTSF EGPFGKIVHQ VRASIDTPRF SKDHKCSLVF YILSPLNLNS
IPDIEQPNVA STTKKFSYKL VKTGNVVLTA STDLRGYVVG QVLRLQADIE NQSGKDTSPV VASLLQKVSY
KAKRWIYDVR TIAEVEGTGV KAWRRAQWQE QILVPALPQS ALPGCSLIHI DYYLQVSMKA PEATVTLPLF
VGNIAVNQTP LSPCPGRESS PGTLSLVVPS APPQEEAEAV ASGPHFSDPV SLSTKSHSQQ QPLSAPLGSV
SVTTTEPWVQ VGSPARHSLH PPLCISIGAT VPYFAEGSAG PVPTTSALIL PPEYSSWGYP YEAPPSYEQS
CGAAGTDLGL IPGS (SEQ ID NO:05)

>gi|244798112IrefINP_848495.2| arrestin domain-containing protein 1 isoform b [Mus musculus]

1 MGRVQLFEIR LSQGRVVYGP GEPLAGTVHL RLGAPLPFRA IRVTCMGSCG VSTKANDGAW
61 VVEESYFNSS LSLADKGSLP AGEHNFPFQF LLPATAPTSF EGPFGKIVHQ VRASIDTPRF
121 SKDHKCSLVF YILSPLNLNS IPDIEQPNVA STTKKFSYKL VKTGNVVLTA STDLRGYVVG
181 QVLRLQADIE NQSGKDTSPV VASLLQVSYK AKRWIYDVRT IAEVEGTGVK AWRRAQWQEQ
241 ILVPALPQSA LPGCSLIHID YYLQVSMKAP EATVTLPLEV GNIAVNQTPL SPCPGRESSP
301 GTLSLVVPSA PPQEEAEAVA SGPHFSDPVS LSTKSHSQQQ PLSAPLGSVS VTTTEPWVQV
361 GSPARHSLHP PLCISIGATV PYFAEGSAGP VPTTSALILP PEYSSWGYPY EAPPSYEQSC
421 GAAGTDLGLI PGS SEQ ID NO: 06)

In extracellular vesicles, e.g., micro-vesicles, of the invention, the TSG101 associating protein is stably associated with an RNA binding protein. The RNA binding protein may vary. In some embodiments, the RNA binding protein is a naturally-occurring protein, or non-naturally- occurring variant thereof, or a non-naturally occurring protein that binds to an RNA, for example, an RNA with a specific sequence or structure. For ease of description, going forward the phrase “RNA binding protein” refers to a naturally occurring RNA-binding protein or an active variant thereof.

In certain embodiments, the RNA binding protein is a trans-activator of transcription (Tat) protein that specifically binds a trans-activating response element (TAR element). The sequences of exemplary Tat proteins are provided in Table 1 of PCT Application Serial No. PCT/US2017/054912 published as WO/2018/067546, where Tat proteins of that may be employed include those sequences as well as variants thereof, e.g., Tat proteins that are at least 60%, 65%, 70%, 75%, 80%, 85%, 90%, 95%, 98%, or 99% identical to those sequences, where in some instances the RNA binding protein has at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, or at least 130 identical contiguous amino acids of any one of those TAT sequences. The RNA binding protein may also be a variant of a Tat protein that is capable of associating with a TAR element. Tat proteins, as well as variants of Tat proteins that bind to a TAR element, are known in the art and have been described previously, for example, in Kamine et al., “Mapping of HIV-1 Tat Protein Sequences Required for Binding to Tar RNA”, Virology 182, 570-577 (1991); and Patel, “Adaptive recognition in RNA complexes with peptides and protein modules” Curr Opin Struct Biol. 1999 Feb;9(1):74-87; the entire contents of each of which are incorporated herein by reference. In some embodiments, the Tat protein is an HIV-1 Tat protein, or variant thereof. In some embodiments, the Tat protein is bovine immunodeficiency virus (BIV) Tat protein, or variant thereof. Further details regarding TAT sequences that may be employed as RNA binding proteins of the invention are provided in PCT Application Serial No. PCT/US2017/054912 published as WO/2018/067546, the disclosure of which is herein incorporated by reference.

In some embodiments, the RNA binding protein is a regulator of virion expression (Rev) protein (e.g., Rev from HIV-1), or variant thereof, that binds to a Rev response element (RRE). Rev proteins are known in the art and are known to the skilled artisan. For example, Rev proteins have been described in Fernandes et al., “The HIV-1 Rev response element: An RNA scaffold that directs the cooperative assembly of a homo-oligomeric ribonucleoprotein complex” RNA Biology 9:1, 6-11; January 2012; Cochrane et al., “The human immunodeficiency virus Rev protein is a nuclear phosphoprotein” Virology 171 (I):264-266, 1989; Grate et al., “Role REVersal: understanding how RRE RNA binds its peptide ligand” Structure. 1997 Jan 15;5(1):7-11; and Patel, “Adaptive recognition in RNA complexes with peptides and protein modules” Curr Opin Struct Biol. 1999 Feb;9(1):74-87; the entire contents of each of which are incorporated herein by reference in their entirety. Further details regarding Rev proteins that may be employed as RNA binding proteins of the invention are provided in PCT Application Serial No. PCT/US2017/054912 published as WO/2018/067546, the disclosure of which is herein incorporated by reference.

In some embodiments, the RNA binding protein is a coat protein of an MS2 bacteriophage that specifically binds to an MS2 RNA. MS2 bacteriophage coat proteins that specifically bind MS2 RNAs are known in the art. For example, MS2 phage coat proteins have been described in Parrott et al., “RNA aptamers for the MS2 bacteriophage coat protein and the wild-type RNA operator have similar solution behavior” Nucl. Acids Res. 28(2) :489-497 (2000); Keryer-Bibens et al., “Tethering of proteins to RNAs by bacteriophage proteins” Biol. Cell. 100(2): 125-38 (2008); and Patel, “Adaptive recognition in RNA complexes with peptides and protein modules” Curr Opin Struct Biol. 1999 Feb;9(1):74-87; the entire contents of each are hereby incorporated by reference in their entirety. Further details regarding MS2 coat proteins that may be employed as RNA binding proteins of the invention are provided in PCT Application Serial No. PCT/US2017/054912 published as WO/2018/067546, the disclosure of which is herein incorporated by reference.

In some embodiments, the RNA binding protein is a P22 N protein (e.g., P22 N from bacteriophage), or variant thereof, that binds to a P22 boxB RNA. P22 N proteins are known in the art and would be apparent to the skilled artisan. For example, P22 N proteins have been described in Cai et al., “Solution structure of P22 transcriptional antitermination N peptide-boxB RNA complex” Nat Struct Biol. 1998 Mar;5(3):203-12; and Patel, “Adaptive recognition in RNA complexes with peptides and protein modules” Curr Opin Struct Biol. 1999 Feb;9(1):74-87; the entire contents of each are incorporated by reference herein. Further details regarding P22 N proteins that may be employed as RNA binding proteins of the invention are provided in PCT Application Serial No. PCT/US2017/054912 published as WO/2018/067546, the disclosure of which is herein incorporated by reference.

In some embodiments, the RNA binding protein is a λ N protein (e.g., λ N from bacteriophage), or variant thereof, that binds to a λ boxB RNA. λ N proteins are known in the art and would be apparent to the skilled artisan. For example, λ N proteins have been described in Keryer-Bibens et al., “Tethering of proteins to RNAs by bacteriophage proteins” Biol Cell. 2008 Feb; 100(2): 125-38; Legault et al., “NMR structure of the bacteriophage lambda N peptide/boxB RNA complex: recognition of a GNRA fold by an arginine-rich motif Cell. 1998 Apr 17;93(2):289-99; and Patel, “Adaptive recognition in RNA complexes with peptides and protein modules” Curr Opin Struct Biol. 1999 Feb;9(1):74-87; the entire contents of each are incorporated by reference herein. Further details regarding λ N proteins that may be employed as RNA binding proteins of the invention are provided in PCT Application Serial No. PCT/US2017/054912 published as WO/2018/067546, the disclosure of which is herein incorporated by reference.

In some embodiments, the RNA binding protein is a φ21 N protein (e.g., φ21 N from bacteriophage), or variant thereof, that binds to a φ21 boxB RNA. φ21 N proteins are known in the art and would be apparent to the skilled artisan. For example, φ21 proteins have been described in Cilley et al. “Structural mimicry in the phage φ21 N peptide-boxB RNA complex.” RNA. 2003;9(6):663-676; and Patel, “Adaptive recognition in RNA complexes with peptides and protein modules” Curr Opin Struct Biol. 1999 Feb;9(1):74-87; the entire contents of each are incorporated by reference herein. Further details regarding φ21 N proteins that may be employed as RNA binding proteins of the invention are provided in PCT Application Serial No. PCT/US2017/054912 published as WO/2018/067546, the disclosure of which is herein incorporated by reference.

In some embodiments, the RNA binding protein is a HIV-1 nucleocapsid (e.g., nucleocapsid from HIV-1), or variant thereof, that binds to a SL3 ψ RNA. HIV-1 nucleocapsid proteins are known in the art and would be apparent to the skilled artisan. For example, HIV-1 nucleocapsid proteins have been described in Patel, “Adaptive recognition in RNA complexes with peptides and protein modules” Curr Opin Struct Biol. 1999 Feb;9(I):74-87; the entire contents of which is incorporated by reference herein. Further details regarding HIV-1 nucleocapsid proteins that may be employed as RNA binding proteins of the invention are provided in PCT Application Serial No. PCT/US2017/054912 published as WO/2018/067546, the disclosure of which is herein incorporated by reference.

As reviewed above, in micro-vesicles of the invention, the TSG101 associating protein is stably associated with the RNA binding protein. In some embodiments the RNA binding protein is covalently linked to the TSG101 associating protein. The RNA binding protein, for example, may be covalently linked to the N-terminus, the C -terminus, or within the amino acid sequence of the TSG101 associating protein. In some embodiments the RNA binding protein is non-covalently linked to the TSG101 associating protein. As such, the TSG101 associating protein may be stably associated with the RNA binding protein, either covalently or non-covalently. Where the TSG101 protein is covalently bound to the RNA binding protein, the TSG101 associating protein may be fused to the RNA binding protein such that the TSG101 associating protein and RNA binding protein are present as a fusion protein. In some aspects, fusion proteins are provided that comprise a TSG101 associating protein fused to a Tat protein. In certain embodiments, the RNA binding protein is fused to the C-terminus of the TSG101 associating protein. The RNA binding protein may also be fused to the N terminus of the TSG101 protein. In some embodiments, the RNA binding protein or RNA binding protein variant may be within the TSG101 protein.

In some embodiments, the RNA binding protein is associated with the TSG101 associating protein via a linker. In some embodiments, the linker is a cleavable linker, for example, the linker may contain a protease recognition site or a disulfide bond. The protease recognition site of the linker may be recognized by a protease expressed in a target cell, resulting in the RNA binding protein fused to the TSG101 protein or variant thereof being released into the cytoplasm of the target cell upon uptake of the micro-vesicle. A person skilled in the art would appreciate that any number of linkers may be used to fuse the RNA binding protein or RNA binding protein variant to the TSG101 associating protein, or variant thereof. When present, the linker may be cleavable or uncleavable. In some embodiments, the linker comprises an amide, ester, ether, carbon-carbon, or disulfide bond, although any covalent bond in the chemical art may be used. In some embodiments, the linker comprises a labile bond, cleavage of which results in separation of the RNA binding protein from the TSG101 associating protein, or variant thereof. In some embodiments, the linker is cleaved under conditions found in the target cell (e.g., a specific pH, a reductive environment, or the presence of a cellular enzyme). In some embodiments, the linker is cleaved by a cellular enzyme. In some embodiments, the cellular enzyme is a cellular protease or a cellular esterase. In some embodiments, the cellular enzyme is a cytoplasmic protease, an endosomal protease, or an endosomal esterase. In some embodiments, the cellular enzyme is specifically expressed in a target cell or cell type, resulting in preferential or specific release of the RNA binding protein in the target cell or cell type. The target sequence of the protease may be engineered into the linker between the RNA binding protein and the TSG101 associating protein, or variant thereof. Additional linkers that may be used in accordance with the disclosure include, without limitation, those described in Chen et al., “Fusion Protein Linkers: Property, Design and Functionality” Adv Drug Deliv Rev. 2013 Oct 15; 65(10): 1357-1369; and Choi et al., “Protease-Activated Drug Development” Theranostics, 2012; 2(2): 156-178; the entire contents of each of which are incorporated herein by reference in their entirety. Other suitable linkers will be apparent to those of skill in the art and are within the scope of this disclosure. Further details regarding HIV-1 linkers that may be employed in embodiments of the invention are provided in PCT Application Serial No. PCT/US2017/054912 published as WO/2018/067546, the disclosure of which is herein incorporated by reference. Any of the linkers, described herein, may be fused to the C-terminus of the TSG101 associating protein, or variant thereof, and the N-terminus of the RNA binding protein, or variant thereof, thereby linking the TSG101 associating protein, or variant thereof, to the RNA binding protein or RNA binding protein variant. In other embodiments, the linker may be fused to the C-terminus of the RNA binding protein, or variant thereof, and the N-terminus of the TSG101 associating protein, or variant thereof.

In some embodiments, any of the fusion proteins or linkers provided herein comprise one or more nuclear localization sequence (NLS). As used herein, a nuclear localization sequence refers to an amino acid sequence that promotes localization of a protein, for example, an RNA binding protein bound to a binding RNA having an NLS, into the nucleus of the cell (e.g., via nuclear transport). Typically, an NLS comprises one or more short amino acid sequences of positively charged lysines or arginines exposed on the protein surface. Nuclear localization sequences are known in the art and would be apparent to the skilled artisan. For example, nuclear localization sequences have been described in Kosugi et al., “Six Classes of Nuclear Localization Signals Specific to Different Binding Grooves of Importin a” J. Biol. Chem. Jan. 2, 2008, 284 p.478-85; Kalderon et al., “A short amino acid sequence able to specify nuclear location” Cell (1984) 39 (3 Pt 2): 499-509; Dingwall et al., “The nucleoplasmin nuclear location sequence is larger and more complex than that of SV-40 large T antigen”. J Cell Biol. (1988) 107 (3): 841-9; Makkerh, et al., “Comparative mutagenesis of nuclear localization signals reveals the importance of neutral and acidic amino acids”. Curr Biol. (1996) 6 (8): 1025-7; and Ray et al., “Quantitative tracking of protein trafficking to the nucleus using cytosolic protein delivery by nanoparticle-stabilized nanocapsules”. Bioconjug. Chem. (2015) 26 (6): 1004-7; the entire contents of each of which are incorporated herein by reference. Additional nuclear localization sequences are described, for example, in Plank et al., international PCT application, PCT/EP2000/011690, the entire contents of which are incorporated herein by reference. In some embodiments, a NLS comprises the amino acid sequence PKKKRKV (SEQ ID NO: 7) or MDSLLMNRRKFLYQFKNVRWAKGRRETYLC (SEQ ID NO: 8).

In some embodiments, the RNA binding protein is fused to at least one NLS. In some embodiments, one or more nuclear localization sequences (NLSs) are fused to the N-terminus of an RNA binding protein. In some embodiments, one or more NLSs are fused to the C-terminus of an RNA binding protein. In some embodiments, an RNA binding protein is fused to at least one, at least two, at least three, at least four, at least five, at least six, at least seven, at least eight, at least nine, at least ten, or more NLSs. It should be appreciated that one or more NLSs may be fused to an RNA binding protein to allow localization of the RNA binding protein into the nucleus of a target cell. In some embodiments, the RNA binding protein fused to at least one NLS is associated with ARRDC1, or an ARRDC1 protein variant.

In some embodiments, any of the fusion proteins or linkers provided herein comprise one or more protein tags, which may be useful for solubilization, purification, or detection of the fusion proteins. In some embodiments, the fusion protein or linker comprises 1, 2, 3, 4, 5, 6, 7, 8, 9, or 10 protein tags. Suitable protein tags are provided herein, and include, without limitation, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione- S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable protein tags will be apparent to those of skill in the art and are within the scope of this disclosure.

Additional details regarding how the TSG101 associating protein may be stably associated with the RNA binding protein provided in PCT Application Serial No. PCT/US2017/054912 published as WO/2018/067546, the disclosure of which is herein incorporated by reference.

### Cargo Prodrug RNA Complexes

Micro-vesicles of the invention include one or more cargo RNA complexes, where the cargo RNA complexes include a cargo RNA stably associated, e.g., covalently bound to, a binding RNA that binds to an RNA binding protein, i.e., an RNA ligand of an RNA binding protein. Cargo RNA complexes may be viewed as having at least two components, i.e., a cargo RNA component and a binding RNA component. The two components may be joined by a linker, which linker may vary in length, ranging in some instances from 1 to 200 nt, such as 10 to 60 nt. In the cargo-RNA complexes, the association of the cargo RNA with the binding RNA serves to protect the cargo RNA from premature processing until the cargo RNA complex is delivered by the micro-vesicle to the target cell. As such, the cargo RNA complexes serve as a type of switch in which, prior to delivery to the target cell, the association of the cargo RNA with the binding RNA protects the cargo RNA from premature degradation or processing, whereas interruption of the association in targeted cells enables the cargo RNA to be processed into an active agent, e.g., siRNA. The cargo RNA component of the cargo RNA complexes may be viewed as an inactive precursor of an RNA having activity that is desired in the target cell for which the micro-vesicle is configured for use. In other words, the cargo RNA is a prodrug cargo RNA, in that it is does not exhibit activity in the micro-vesicle producing cell in which the micro-vesicle is produced but, once introduced into the target cell for which the micro-vesicle is configured for use, is processed by one or more components present in the target cell, e.g., Dicer, Drosha, or other RNA processing protein, to produce an RNA, e.g., siRNA, miRNA, etc., having a desired activity in the target cell. Put another way, prodrug cargo RNA does not exhibit activity in the micro-vesicle producing cell because it not processed in in the micro-vesicle producing cell into a form that exhibits activity in the micro-vesicle producing cell. However, once the cargo prodrug RNA is introduced into the target cell for which the micro-vesicle is configured for use, it is processed by one or more components present in the target cell, e.g., Dicer, Drosha, or other RNA processing protein, to produce an RNA, e.g., siRNA, miRNA, etc., having a desired activity in the target cell. Because the cargo RNA is produced in prodrug form in the micro-vesicle producer cells, it may be directed to a target that is exogenous or endogenous to the producer cells, since it will not exhibit activity in the producer cells. Furthermore, because the cargo prodrug RNA is not processed in the micro-vesicle producing cell, the cargo prodrug RNA can be efficiently delivered into the micro-vesicle by the ARDDC1-Tat part of the complex (since processing of the prodrug RNA would separate all or a portion of the prodrug RNA from the binding RNA component, e.g., Tar, thereby preventing or reducing prodrug RNA delivery into the micro-vesicle). Embodiments of the invention provide further advantages by having the cargo RNA in prodrug form in the micro-vesicle producing cell. For example, processing and activation of the prodrug RNA in the micro-vesicle producing cell could affect functions of the producing cell. For example, activation of the prodrug RNA in the producing cell could decrease the viability of the micro-vesicle producing cell by knocking down the ability of the micro-vesicle producing cell to make a needed gene product, e.g., Supt4h. In yet other instances, processing of the prodrug RNA in the producing cell could create a form that is rendered non-functional by degradation, which may be undesirable where one wants to isolate the prodrug cargo RNA and use other compositions, such as lipid nanoparticles, gold nanoparticles, exosomes, etc., to introduce the cargo RNA into cells. The micro-vesicles of embodiments of the present invention overcome these disadvantages by providing the cargo RNA in prodrug format that is not processed in the micro-vesicle producing cell.

The number of distinct cargo RNA complexes that differ in sequence at least with respect to their cargo RNA component present in the micro-vesicles may vary. In some instances, the micro-vesicles include a single type of cargo RNA complex. In yet other embodiments, the micro-vesicles include a plurality of two or more distinct cargo RNA complexes that differ from each other at least with respect to the nucleotide sequences of their cargo RNA components. In these embodiments, individual micro-vesicles or a population of micro-vesicles that are produced by a single clonal population of cells includes a plurality of distinct cargo RNAs each containing different domains that target different sequences of a target, such as a viral RNA target. As such, the number of distinct cargo RNA complexes in a given micro-vesicle of the invention may vary, ranging in some instances from 1 to 20, such as 1 to 15, including 1 to 10, e.g., 1 to 5. In those embodiments where the micro-vesicle includes a plurality of distinct cargo RNAs, in some instances the micro-vesicle includes 2 to 10, such as 2 to 5, distinct cargo RNAs each containing different RNA domains that target different sequences of a target, such as a viral RNA target. In some instances, the cargo RNA complexes include an RNA cargo component covalently attached to a binding RNA that binds non-covalently to an RNA binding protein. In those instances where the extracellular vesicle, e.g., micro-vesicle, includes a plurality of distinct cargo RNA complexes that differ from each other in terms of their cargo RNA components, the binding RNA components of the cargo RNA complexes may be the same or different. As such, in some instances where a plurality of distinct cargo RNA complexes is present, the binding RNA components of the complexes may be identical. The target of the cargo RNA may vary, and may be a nucleic acid, e.g., viral RNA, a protein, e.g., a viral protein, or a metabolite, e.g., a viral metabolite.

Cargo RNA components of the cargo RNA complexes present in the micro-vesicles of the invention may vary. In some instances, the RNA component is a prodrug or inactive precursor of an inhibitory ribonucleic acid. The inhibitory RNA may be interfering RNA, (i.e., RNAi), an antisense RNA, a ribozyme, or combinations thereof. The RNA component may be a small interfering RNA or siRNAs, a small hairpin RNA or shRNAs, microRNA or miRNAs, a double-stranded RNA (dsRNA), etc. The RNA component may be a precursor of a short RNA molecule, such as a short interfering RNA (siRNA), a small temporal RNA (stRNA), and a micro-RNA (miRNA). The inhibitory RNA may be an antisense RNA. In yet other instances, the inhibitory RNA may be a ribozyme. In some embodiments, the inhibitory RNA is selected from the group consisting of a short interfering RNA (siRNA), an asymmetrical interfering RNA (aiRNA), an RNA interference (RNAi) molecule, a microRNA (miRNA), an antagomir, an antisense RNA, a ribozyme, a Dicer-substrate RNA (dsRNA), a small hairpin RNA (shRNA), a Drosha sensitive RNA, an RNA that is cleaved by another RNA processing protein, and combinations thereof.

In some instances, the cargo RNA components may be viewed as siRNA precursors, which may also be referred to as RNAi prodrugs, which precursors include a domain or region which can be processed into a siRNA that binds to a viral RNA target sequence. In some instances, the precursor domain is a double-stranded domain that can be cleaved by Dicer (i.e., Dicer 1, Ribonuclease III) to produce an active siRNA. While the length of this double-stranded precursor domain may vary, in some instances the length is 30 nt or longer, ranging in some instances from 30 to 100 nt, such as 40 to 80 nt. In some instances, the double-stranded precursor domain includes two complementary hybridized strands joined by a loop, such that the double-stranded precursor domain may be viewed as having a short hairpin structure (and may be referred to herein as an shRNA domain). In such instances, the length of the hybridized region may vary, ranging in some instances from 15 to 25 nt, such as 20 to 25 nt. The length of the loop may also vary, ranging in some instances from 5 to 30 nt, such as 5 to 15 nt, e.g., 5 to 10 nt. In some instances, the cargo ribonucleic acid complexes include a short hairpin RNA (shRNA) domain that targets a sequence of a target RNA, such as viral RNA; and an RNA that is a ligand for an RNA binding protein.

In yet another example, the prodrug cargo RNA may be an RNA incapable of replication in the micro-vesicle producing cell but capable of binding products made by an infectious viral pathogen in the targeted cell, e.g., where the cargo prodrug RNA is comprised of nucleotides that represent part but not all or a replicon, such that it may be viewed as a defective replicon of an infectious viral pathogen being treated. In such instances, the decoy RNA that may be viewed as a defective replicon is used as a “decoy” in the target cell to bind a viral RNA polymerase or another gene product made by the pathogenic virus infecting the targeted cell. As such, in some instances, the cargo prodrug RNA may be a precursor of a decoy RNA. The term decoy RNA is used to refer to an RNA that, when present in the target cell for which the micro-vesicle is configured for use, is processed by an activity in the target cell, such as a viral polymerase, in a manner that achieves a desired effect, e.g., a therapeutic effect. For example, a decoy RNA may be a decoy for a viral polymerase, e.g., where the decoy RNA is similar to a genuine viral RNA but cannot be processed by a viral polymerase into a functional product. In such embodiments, upon delivery of the decoy RNA to a target cell infected with a virus for which the decoy RNA is designed to target, the decoy RNA may compete with viral RNA for processing by an RNA polymerase of the virus. By having the decoy RNA encode a product that inhibits production of infectious viral particles, the decoy RNA provides a desired, therapeutic effect in inhibiting infection viral particle production. For example, a micro-vesicle may deliver multiple copies of a cargo precursor decoy RNA that lacks sequences required to make infectious viral particles, but still competes with its corresponding fully functional virus RNA for the polymerase to reduce or inhibit the production of infectious viral particles. In yet another embodiment, instead of or in addition to such decoy RNAs, the decoy RNA may be provided that competes with some other genuine viral RNA that that encodes an essential viral protein in order to reduce or inhibit production of infectious viral particles. Where desired, such decoy RNAs may be further operably linked to RNA encoding a toxin, such that processing of the decoy RNA n virus-infected cells produces a toxin that kills the target cell. In such embodiments, cells lacking the viral polymerase would not process the decoy RNA because they lack the viral polymerase and therefore would not produce toxin.

The cargo RNA may be configured to modulate a variety of different types of targets. In some instances, the target is an RNA target, e.g., an RNA target involved in, i.e., that mediates, a target disease condition. In embodiments where the RNA target is one that mediates a disease condition of a subject, the RNA target may be endogenous to the subject or exogenous to the subject, e.g., there the RNA target a pathogenic target, such as a viral target. As such, in some instances the target RNA is a non-viral RNA, such as a target RNA that is an endogenous target RNA to a host for which the micro-vesicle is configured for use, e.g., an mRNA encoding a product that mediates a disease condition to be treated. As reviewed above, as the cargo RNA is a prodrug when generated and packaged in the micro-vesicle by the producer cells, the target RNA may be endogenous to the producer cells. In other embodiments, the target RNA is an exogenous RNA, such as a pathogenic RNA, e.g., a viral, bacterial, yeast or fungal RNA.

As described above, in some embodiments the cargo-RNA component of the cargo RNA complexes in micro-vesicles of embodiments of the invention includes a sequence that targets a sequence of a target viral RNA. As such, the cargo-RNA component includes a viral targeting RNA sequence that hybridizes to a target sequence present in a target viral RNA. The specific sequence of the viral targeting RNA sequence may vary depending on the nature of the target viral RNA. The target viral RNA may be an RNA of a variety of different types of target viruses. In general, target viruses may be any viruses that infect cells that may be contacted by micro-vesicles of the invention. Viruses that may be targeted may vary, where target viruses include, but are not limited to, pathogenic viruses, such as viruses associated with, e.g., cause, respiratory diseases, such as but not limited to, coronaviruses, influenza viruses, and the like

In some instances, the target virus is a coronavirus. Coronaviruses that may be targeted by cargo RNAs of the invention include, but are not limited to: SARS-CoV-2, SARS-CoV, MERS-CoV, HCoV-OC43, HCoV-229E, HCoV-HKU1 and HCoV-NL63, etc. Target viral RNA sequences may vary, where examples of target viral RNA sequences include those found in coding regions, where coding regions of interest include, but are not limited to, coding regions for proteins essential to virus propagation or that are associated with viral infection detrimental effects, where specific coding regions of interest include, but are not limited to, coding regions for RNA-dependent RNA polymerase (RdRP), etc.

Where the target virus is SARS-CoV-2 and the viral target RNA encodes RdRP, any convenient subsequence of the coding sequence may be bound by the viral targeting RNA of the cargo-RNA component. Specific RdRP subsequences that may be targeted include, but are not limited to: shRDRP-1: AGGAAGTTCTGTTGAATTAAA (SEQ ID NO:9) and shRDRP-8: CTGCATTGTGCAAACTTTAAT(SEQ ID NO:10)

The binding RNA component (i.e., ligand RNA) of the cargo RNA complexes which binds to the RNA binding protein may vary, as desired. In some instances, the binding RNA is a naturally occurring RNA, or non-naturally occurring variant thereof, or a non-naturally occurring RNA, that binds to a protein having a specific amino acid sequence or structure. In certain embodiments, the binding RNA is a trans-activating response element (TAR element), which is an RNA stem-loop structure that is found at the 5′ ends of nascent human immunodeficiency virus- 1 (HIV-1) transcripts and specifically bind to a trans- activator of transcription (Tat) protein. In some embodiments, the TAR element is a bovine immunodeficiency virus (BIV) TAR. Exemplary TAR sequences that may be employed in embodiments of the invention can be found in Table 2 of PCT Application Serial No. PCT/US2017/054912 published as WO/2018/067546, the disclosure of which is herein incorporated by reference. The binding RNA may also be a variant of a TAR element that is capable of associating with the RNA binding protein, trans-activator of transcription (Tat protein), which is a regulatory protein that is involved in transcription of the viral genome. Variants of TAR elements that are capable of associating with Tat proteins would be apparent to the skilled artisan based on this disclosure and knowledge in the art, and are within the scope of this disclosure. Further, the association between a TAR variant and a Tat protein, or Tat protein variant, may be tested using routine methods. TAR elements and variants of TAR elements that bind to Tat proteins are known in the art and have been described previously, for example in Kamine et al., “Mapping of HIV-1 Tat Protein Sequences Required for Binding to Tar RNA” Virology 182, 570-577 (1991); and Patel, “Adaptive recognition in RNA complexes with peptides and protein modules” Curr Opin Struct Biol. 1999 Feb;9(1):74-87; the entire contents of each are incorporated by reference herein.

In some embodiments, the binding RNA is a Rev response element (RRE), or variant thereof, that binds to a Rev protein (e.g., Rev from HIV-1). Rev response elements are known in the art and would be apparent to the skilled artisan for use in the present invention. For example, Rev response elements have been described in Fernandes et al., “The HIV-1 Rev response element: An RNA scaffold that directs the cooperative assembly of a homo-oligomeric ribonucleoprotein complex.” RNA Biology 9:1, 6-11, January 2012; Cook et al., “Characterization of HIV-1 REV protein: binding stoichiometry and minimal RNA substrate.” Nucleic Acids Res. Apr 11; 19(7): 1577-1583, 1991; Grate et al., “Role REVersal: understanding how RRE RNA binds its peptide ligand” Structure. 1997 Jan 15;5(1):7-11; and Patel, “Adaptive recognition in RNA complexes with peptides and protein modules” Curr Opin Struct Biol. 1999 Feb;9(1):74-87; the entire contents of each are incorporated herein by reference. Any of the RRE nucleic acid sequences or any of the fragments of RRE nucleic acid sequences described in the above references may be used as binding RNAs in accordance with this disclosure. Further details regarding exemplary RRE nucleic acid sequences that bind Rev include, without limitation, those nucleic acid sequences provided in PCT Application Serial No. PCT/US2017/054912 published as WO/2018/067546, the disclosure of which is herein incorporated by reference.

In some embodiments, the binding RNA is an MS2 RNA that specifically binds to a MS2 phage coat protein. Typically, the coat protein of the RNA bacteriophage MS2 binds a specific stem-loop structure in viral RNA (e.g., MS2 RNA) to accomplish encapsidation of the genome and translational repression of replicase synthesis. RNAs that specifically bind MS2 phage coat proteins are known in the art and would be apparent the skilled artisan. For example, RNAs that bind MS2 phage coat proteins have been described in Parrott et al., “RNA aptamers for the MS2 bacteriophage coat protein and the wild-type RNA operator have similar solution behavior.” Nucl. Acids Res. 28(2): 489-497 (2000); Witherell et al., “Specific interaction between RNA phage coat proteins and RNA.” Prog Nucleic Acid Res Mol Biol. 1991;40:185-220; Stockley et al., “Probing sequence-specific RNA recognition by the bacteriophage MS2 coat protein.” Nucleic Acids Res. 1995 Jul 11;23(13):2512-8; Keryer-Bibens C, et al., “Tethering of proteins to RNAs by bacteriophage proteins.” Biol. Cell. 100(2): 125-38 (2008); and Patel. “Adaptive recognition in RNA complexes with peptides and protein modules.” Curr Opin Struct Biol. 1999 Feb;9(1):74-87; the entire contents of each are hereby incorporated by reference in their entirety. In some embodiments, an exemplary MS2 RNA that specifically binds to a MS2 phage coat protein comprises a nucleic acid sequence as provided in PCT Application Serial No. PCT/US2017/054912 published as WO/2018/067546, the disclosure of which is herein incorporated by reference.

In some embodiments, the binding RNA is an RNA that specifically binds to a P22 N protein (e.g., P22 N from bacteriophage), or variant thereof. P22 N proteins are known in the art and would be apparent to the skilled artisan. For example, P22 N proteins have been described in Cai et al., “Solution structure of P22 transcriptional antitermination N peptide-boxB RNA complex” Nat Struct Biol. 1998 Mar;5(3):203-12; Weiss, “RNA- mediated signaling in transcription” Nat Struct Biol. 1998 May;5(5):329-33; and Patel, “Adaptive recognition in RNA complexes with peptides and protein modules” Curr Opin Struct Biol. 1999 Feb;9(1):74-87; the entire contents of each are incorporated by reference herein. Further details regarding such sequences are provided in PCT Application Serial No. PCT/US2017/054912 published as WO/2018/067546, the disclosure of which is herein incorporated by reference.

In some embodiments, the binding RNA is an RNA that specifically binds to a λ N protein (e.g., λ N from bacteriophage), or variant thereof, λ N proteins are known in the art and would be apparent to the skilled artisan. For example, λ N proteins have been described in Keryer-Bibens et al., “Tethering of proteins to RNAs by bacteriophage proteins.” Biol Cell. 2008 Feb; 100(2): 125-38; Weiss. “RNA-mediated signaling in transcription.” Nat Struct Biol. 1998 May;5(5):329-33; Legault et al., “NMR structure of the bacteriophage lambda N peptide/boxB RNA complex: recognition of a GNRA fold by an arginine-rich motif.” Cell.1998 Apr 17;93(2):289-99; and Patel, “Adaptive recognition in RNA complexes with peptides and protein modules.” Curr Opin Struct Biol. 1999 Feb;9(I):74-87; the entire contents of each are incorporated by reference herein. An exemplary λ boxB RNA that specifically binds to a λ N protein comprises a nucleic acid sequence as set forth in gggcccugaagaagggccc (SEQ ID NO:11), where further details regarding such sequences are provided in PCT Application Serial No. PCT/US2017/054912 published as WO/2018/067546, the disclosure of which is herein incorporated by reference.

In some embodiments, the binding RNA is an RNA that specifically binds to a φ21 N protein (e.g., φ21 N from bacteriophage), or variant thereof. φ21 N proteins are known in the art and would be apparent to the skilled artisan. For example, φ21 proteins have been described in Cilley et al. “Structural mimicry in the phage φ21 N peptide-boxB RNA complex.” RNA. 2003;9(6):663-676; and Patel, “Adaptive recognition in RNA complexes with peptides and protein modules.” Curr Opin Struct Biol. 1999 Feb;9(1):74-87; the entire contents of each are incorporated by reference herein. An exemplary φ21 boxB RNA that specifically binds to a φ21 N protein comprises a nucleic acid sequence as set forth in ucucaaccuaaccguugaga (SEQ ID NO:12), where further details regarding such sequences are provided in PCT Application Serial No. PCT/US2017/054912 published as WO/2018/067546, the disclosure of which is herein incorporated by reference.

In some embodiments, the binding RNA is an RNA that specifically binds to an HIV-1 nucleocapsid protein (e.g., nucleocapsid from HIV-1) or variant thereof. HIV-1 nucleocapsid proteins are known in the art and would be apparent to the skilled artisan. For example, HrV-1 nucleocapsid proteins have been described in Patel, “Adaptive recognition in RNA complexes with peptides and protein modules.” Curr Opin Struct Biol. 1999 Feb;9(I):74-87; the entire contents of which is incorporated by reference herein. An exemplary SL3 _{Ψ} RNA that specifically binds to a HIV-1 nucleocapsid comprises a nucleic acid sequence as set forth in ggacuagcggaggcuagucc(SEQ ID NO:13), where further details regarding such sequences are provided in PCT Application Serial No. PCT/US2017/054912 published as WO/2018/067546, the disclosure of which is herein incorporated by reference.

It should be appreciated that the binding RNAs of the present disclosure need not be limited to naturally-occurring RNAs or non-naturally-occurring variants thereof, that have recognized protein binding partners. In some embodiments, the binding RNA may also be a synthetically produced RNA, for example an RNA that is designed to specifically bind to a protein (e.g., an RNA binding protein). In some embodiments, the binding RNA is designed to specifically bind to any protein of interest, for example ARRDC1. In some embodiments, the binding RNA is an RNA produced by the systematic evolution of ligands by exponential enrichment (SELEX). SELEX methodology would be apparent to the skilled artisan and has been described previously, for example in U.S. Pat. Nos. 5,270,163; 5,817,785; 5,595,887; 5,496,938; 5,475,096; 5,861,254; 5,958,691; 5,962,219; 6,013,443; 6,030,776; 6,083,696; 6,110,900; 6,127,119; and 6,147,204; U.S. Appln 20030175703 and 20030083294, Potti et al., Expert Opin. Biol. Ther. 4:1641-1647 (2004), and Nimjee et al., Annu. Rev. Med. 56:555-83 (2005). The technique of SELEX has been used to evolve aptamers to have extremely high binding affinity to a variety of target proteins. See, for example, Trujillo U. H., et al., “DNA and RNA aptamers: from tools for basic research towards therapeutic applications”. Comb Chem High Throughput Screen 9 (8): 619-32 (2006) for its disclosure of using SELEX to design aptamers that bind vascular endothelial growth factor (VEGF). In some embodiments, the binding RNA is an aptamer that specifically binds a target protein, for example a protein found in an ARMM (e.g., ARRDC1 or TSG101).

In some embodiments, any of the cargo RNA components provided herein are stably associated with a binding RNA component. In some embodiments, the cargo RNA component is covalently associated with the binding RNA component. In some embodiments, the cargo RNA and the binding RNA are part of the same RNA molecule, (e.g., an RNA from a single transcript). In some embodiments, the cargo RNA and the binding RNA are covalently associated via a linker. In some embodiments, the linker comprises a nucleotide or nucleic acid (e.g., DNA or RNA). In some embodiments, the linker comprises RNA. In some embodiments, the linker comprises at least 1, at least 2, at least 3, at least 4, at least 5, at least 6, at least 7, at least 8, at least 9, at least 10, at least 15, at least 20, at least 25, at least 30, at least 40, at least 50, at least 100, at least 150, at least 200, at least 250, at least 300, at least 400, or at least 500 nucleotides (e.g., DNA or RNA).

In other embodiments, the cargo RNA is non-covalently associated with the binding RNA. For example, the cargo RNA may associate with the binding RNA via complementary base pairing. In some embodiments, the cargo RNA is bound to the binding RNA via at least 2, at least 3, at least 4, at least 5, at least 6, at least 7, at least 8, at least 9, at least 10, at least 15, at least 20, at least 25, at least 30, at least 40, at least 50, complementary base pairs, which may be contiguous or non-contiguous. In some embodiments, the cargo RNA is bound to the binding RNA via at least 2, at least 3, at least 4, at least 5, at least 6, at least 7, at least 8, at least 9, at least 10, at least 15, at least 20, at least 25, at least 30, at least 40, at least 50 contiguous complementary base pairs.

Any of the RNAs provided herein (e.g., binding RNAs, cargo RNAs, and/or binding RNAs fused to cargo RNAs) may comprise one or more modified oligonucleotides. In some embodiments, any of the RNAs described herein may be modified, e.g., comprise a modified sugar moiety, a modified internucleoside linkage, a modified nucleotide and/or combinations thereof. In some embodiments, RNA oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O--N-methylacetamido (2′-O--NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom. Any of the modified chemistries or formats of RNA oligonucleotides described herein can be combined with each other, and that one, two, three, four, five, or more different types of modifications can be included within the same molecule. In some embodiments, the RNA oligonucleotide may comprise at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States Pat. or Pat. Application Publications: US 7,399,845, US 7,741,457, US 8,022,193, US 7,569,686, US 7,335,765, US 7,314,923, US 7,335,765, and US 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

### Expression Constructs

Some aspects of this invention provide expression constructs that encode any of the fusion proteins described herein. For example, the expression constructs may encode an RNA binding protein fused to a TSG101 associating protein (e.g., ARRDC1:Tat) or an RNA binding protein fused to one or more WW domains. In some embodiments, the expression constructs described herein may further encode, or encode separately, a binding RNA. The binding RNA may be expressed under the control of the same promoter sequence or a different promoter sequence as any of the fusion proteins described herein. In some embodiments, an expression construct encoding a binding RNA is co-expressed with any of the expression constructs described herein. In some embodiments, the expression constructs described herein may further encode, or encode separately, a cargo RNA component or complex thereof. In some embodiments, the cargo RNA component is expressed under the control of the same promoter sequence or a different promoter sequence as any of the fusion proteins or binding RNAs provided herein. In some embodiments, the cargo RNA component is expressed as part of the same transcript as the binding RNA component. For example, the binding RNA and the cargo RNA may be expressed as a single transcript. In some embodiments, the construct encodes a cargo RNA that is fused 5′ to the binding RNA. In some embodiments, the construct encodes a cargo RNA that is fused 3′ to the binding RNA. In some embodiments, the construct encodes a cargo RNA and a binding RNA that are fused via one or more linkers. It should be appreciated that the cargo RNA may also be expressed as a separate transcript from the binding RNA. When expressed as a separate transcript, the cargo RNA may comprise a sequence that binds to the binding RNA (e.g., via complementary base pairing). Accordingly, in some embodiments, the construct encodes a cargo RNA that may comprise a nucleotide sequence that is complementary to a sequence of a binding RNA. In some embodiments, the cargo RNA is expressed from a separate expression construct from the construct encoding the RNA binding protein and/or the binding RNA. In some embodiments, the cargo RNA is expressed from the same construct (e.g., expression vector) encoding the RNA binding protein and/or the binding RNA, but under a different promoter.

In some embodiments, the expression constructs described herein may further encode a gene product or gene products that induce or facilitate the generation of micro-vesicles, e.g., ARMMs, in cells harboring such a construct. In some embodiments, the expression constructs encode an TSG101 associating protein, or variant thereof, and/or a TSG101 protein, or variant thereof. In some embodiments, overexpression of either or both of these gene products in a cell increases the production of micro-vesicles, e.g., ARMMs, in the cell, thus turning the cell into a micro-vesicle producing cell. In some embodiments, such an expression construct comprises at least one restriction or recombination site that allows in-frame cloning of an RNA binding protein sequence to be fused, either at the C-terminus, or at the N-terminus of the encoded TSG101 associating protein, or variant thereof. As another example an expression construct comprises at least one restriction or recombination site that allows in-frame cloning of an RNA binding protein sequence to be fused either at the C-terminus, or at the N-terminus of one re more encoded WW domains.

In some embodiments, the expression construct comprises (a) a nucleotide sequence encoding an TSG101 associating protein operably linked to a heterologous promoter, and (b) a restriction site or a recombination site positioned adjacent to the TSG101 associating protein-encoding nucleotide sequence allowing for the insertion of an RNA binding protein or RNA binding protein variant sequence in frame with the TSG101-encoding nucleotide sequence. In certain embodiments, the expression constructs encode a fusion protein comprising an ARRDC 1 protein and a Tat protein.

Some aspects of this invention provide expression constructs that encode any of the binding RNAs, cargo RNAs, or fusions of any of the binding RNAs and cargo RNAs described herein. In some embodiments, the expression construct comprises (a) a nucleotide sequence encoding a binding RNA operably linked to a heterologous promoter, and (b) a restriction site or a recombination site positioned adjacent to the binding RNA-encoding nucleotide sequence allowing for the insertion of a cargo RNA-encoding nucleotide sequence. In some embodiments, the expression construct comprises (a) a nucleotide sequence encoding a cargo RNA operably linked to a heterologous promoter, and (b) a restriction site or a recombination site positioned adjacent to the cargo RNA-encoding nucleotide sequence allowing for the insertion of a binding RNA-encoding nucleotide sequence. In certain embodiments, the expression constructs encode a TAR binding RNA, or variant thereof fused to a cargo RNA.

Nucleic acids encoding any of the fusion proteins, binding RNAs, and/or cargo RNAs, described herein, may be in any number of nucleic acid “vectors”, including those known in the art. As used herein, a “vector” means any nucleic acid or nucleic acid-bearing particle, cell, or organism capable of being used to transfer a nucleic acid into a host cell. The term “vector” includes both viral and nonviral products and means for introducing the nucleic acid into a cell. A “vector” can be used in vitro, ex vivo, or in vivo. Non-viral vectors include plasmids, cosmids, artificial chromosomes (e.g., bacterial artificial chromosomes or yeast artificial chromosomes) and can comprise liposomes, electrically charged lipids (cytofectins), DNA-protein complexes, and biopolymers, for example. Viral vectors include retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences for production of a virus capable of infecting, entering, or being introduced to a cell to deliver nucleic acid therein.

Expression of any of the fusion proteins, binding RNAs, and/or cargo RNAs, described herein, may be controlled by any regulatory sequence (e.g., a promoter sequence), including those known in the art. Regulatory sequences, as described herein, are nucleic acid sequences that regulate the expression of a nucleic acid sequence. A regulatory or control sequence may include sequences that are responsible for expressing a particular nucleic acid (e.g., a ARRDC1:Tat fusion protein) or may include other sequences, such as heterologous, synthetic, or partially synthetic sequences. The sequences can be of eukaryotic, prokaryotic or viral origin that stimulate or repress transcription of a gene in a specific or non-specific manner and in an inducible or non-inducible manner. Regulatory or control regions may include origins of replication, RNA splice sites, introns, chimeric or hybrid introns, promoters, enhancers, transcriptional termination sequences, poly A sites, locus control regions, signal sequences that direct the polypeptide into the secretory pathways of the target cell, and introns. A heterologous regulatory region is not naturally associated with the expressed nucleic acid it is linked to. Included among the heterologous regulatory regions are regulatory regions from a different species, regulatory regions from a different gene, hybrid regulatory sequences, and regulatory sequences that do not occur in nature, but which are designed by one of ordinary skill in the art.

The term operably linked refers to an arrangement of sequences or regions wherein the components are configured so as to perform their usual or intended function. Thus, a regulatory or control sequence operably linked to a coding sequence is capable of affecting the expression of the coding sequence. The regulatory or control sequences need not be contiguous with the coding sequence, so long as they function to direct the proper expression or polypeptide production. Thus, for example, intervening untranslated but transcribed sequences can be present between a promoter sequence and the coding sequence and the promoter sequence can still be considered operably linked to the coding sequence. A promoter sequence, as described herein, is a DNA regulatory region a short distance from the 5′ end of a gene that acts as the binding site for RNA polymerase. The promoter sequence may bind RNA polymerase in a cell and/or initiate transcription of a downstream (3′ direction) coding sequence. The promoter sequence may be a promoter capable of initiating transcription in prokaryotes or eukaryotes. Some non-limiting examples of eukaryotic promoters include the cytomegalovirus (CMV) promoter, the chicken β-actin (CBA) promoter, and a hybrid form of the CBA promoter (CBh).

### Micro-Vesicle Producing Cells

A micro-vesicle-producing cell of the present invention may be a cell containing any of the expression constructs, any of the fusion proteins, any of the binding RNAs, any of the cargo RNAs, and/or any of the binding RNAs fused to any of the cargo RNAs described herein. For example, an inventive micro-vesicle-producing cell may contain one or more recombinant expression constructs encoding (1) a TSG101 associating protein and (2) an RNA binding protein (e.g., a Tat protein), that is associated with the TSG101 associating protein. In certain embodiments, an expression construct in the micro-vesicle-producing cell encodes a binding RNA that associates (e.g., binds specifically) with the RNA binding protein. In some embodiments, an expression construct in the micro-vesicle-producing cell encodes a cargo RNA that associates with the binding RNA. For example, the construct may encode a binding RNA that is fused to a cargo RNA. In some embodiments, the micro-vesicle-producing cell may express a binding RNA and a cargo RNA from different expression constructs or express a binding RNA and a cargo RNA under the control of different promoters.

Micro-vesicle-producing cells according to embodiments of the invention are incapable of separating the binding RNA component from the cargo RNA component and/or of processing the cargo RNA component to produce a siRNA from the siRNA precursor cargo RNA. In embodiments where the precursor siRNA cargo RNA is a double-stranded RNA, such as a shRNA, that is cleaved by Dicer in the target cell to produce an active siRNA, the micro-vesicle-producing cell may be deficient in sufficient Dicer activity to separate cargo RNA from binding RNA or to process the precursor siRNA cargo RNA into active siRNA. Any convenient Dicer deficient cell may be employed, wherein examples of Dicer deficient cells include, but are not limited to, those Dicer deficient host cells described in: Bogerd et al., RNA (2014) 20:923-937; Kanellopoulou et al., Genes Dev. (2005) 19:489-501 and Song & Rossi, “The effect of Dicer knockout or Drosha kmnockout on sRNA interference using various Dicer or Drosha substrates that when processed yield interfering RNA structures,” (doi: https://doi.org/10.1101/2020.04.19.049817). Micro-vesicles produced by such cells will lack functional dicer, or other RNA processing activity, in contrast to micro-vesicles produced by cells that are not dicer deficient. In some embodiments, the micro-vesicle-producing cells are deficient in Drosha activity (see e.g., Kim et al., PNAS Mar. 29, 2016 113 (13) E1881-E1889). Micro-vesicles produced by such cells will lack functional Drosha activity, in contrast to micro-vesicles produced by cells that are not Drosha deficient.

Any of the expression constructs, described herein, may be stably inserted into the genome of the cell. In some embodiments, the expression construct is maintained in the cell, but not inserted into the genome of the cell. In some embodiments, the expression construct is in a vector, for example, a plasmid vector, a cosmid vector, a viral vector, or an artificial chromosome. In some embodiments, the expression construct further comprises additional sequences or elements that facilitate the maintenance and/or the replication of the expression construct in the micro-vesicle-producing cell, or that improve the expression of the fusion protein in the cell. Such additional sequences or elements may include, for example, an origin of replication, an antibiotic resistance cassette, a polyA sequence, and/or a transcriptional isolator. Some expression constructs suitable for the generation of micro-vesicle-producing cells according to aspects of this invention are described elsewhere herein. Methods and reagents for the generation of additional expression constructs suitable for the generation of micro-vesicle producing cells according to aspects of this invention will be apparent to those of skill in the art based on the present disclosure. In some embodiments, the micro-vesicle-producing cell is a mammalian cell, for example, a mouse cell, a rat cell, a hamster cell, a rodent cell, or a nonhuman primate cell. In some embodiments, the micro-vesicle-producing cell is a human cell.

One skilled in the art may employ any convenient technique, including conventional techniques, such as molecular or cell biology, virology, microbiology, and recombinant DNA techniques. Exemplary techniques are explained fully in the literature. For example, one may rely on the following general texts to make and use the invention: Sambrook et al., Molecular Cloning: A Laboratory Manual, Second Edition (1989) Cold Spring Harbor Laboratory Press, Cold Spring Harbor, New York, and Sambrook et al. Third Edition (2001); DNA Cloning: A Practical Approach, Volumes I and II (D.N. Glover ed. 1985); Oligonucleotide Synthesis (M.J. Gaited. 1984); Nucleic Acid Hybridization (B.D. Hames & S.J. Higgins eds. (1985)); Transcription And Translation Hames & Higgins, eds. (1984); Animal Cell Culture (RI.Freshney, ed. (1986)); Immobilized Cells And Enzymes (IRL Press, (1986)); Gennaro et al. (eds.) Remington’s Pharmaceutical Sciences, 18th edition; B. Perbal, A Practical Guide To Molecular Cloning (1984); F.M. Ausubel et al. (eds.), Current Protocols in Molecular Biology, John Wiley & Sons, Inc. (updates through 2001), Coligan et al. (eds.), Current Protocols in Immunology, John Wiley & Sons, Inc. (updates through 2001); W. Paul et al. (eds.) Fundamental Immunology, Raven Press; E.J. Murray et al. (ed.) Methods in Molecular Biology: Gene Transfer and Expression Protocols, The Humana Press Inc. (1991)(especially vol.7); and J.E. Celis et al., Cell Biology: A Laboratory Handbook, Academic Press (1994). The micro-vesicle producing cells may be employed to produce micro-vesicles of the invention using any convenient protocol. In some instances, the micro-vesicle producing cells are maintained under conditions, e.g., nutrient, temperature, etc., sufficient to for the cells to generate micro-vesicles. Such conditions may include those provided in U.S. Pat. Nos. 9,816,080 and 10,260,055, as well as Nabhan et al., PNAS (2012) 109 (11) 4146-4151; the disclosures of which are herein incorporated by reference. Methods for isolating the micro-vesicles described herein are also provided, where details regarding such methods may be found in U.S. Pat. Nos. 9,816,080 and 10,260,055, as well as Nabhan et al., PNAS (2012) 109 (11) 4146-4151; the disclosures of which are herein incorporated by reference.

### Targeting Moieties & Labels

Micro-vesicles (such as ARMMs) of embodiments of the invention containing any of the expression constructs, any of the fusion proteins, any of the binding RNAs, any of the cargo RNAs, and/or any of the binding RNAs fused to any of the cargo RNAs, described herein, may further have a targeting moiety. The targeting moiety may be used to target the delivery of micro-vesicle to specific cell types, resulting in the release of the contents of the micro-vesicle into the cytoplasm of the specific targeted cell type. A targeting moiety may selectively bind an antigen of the target cell. For example, the targeting moiety may be a membrane-bound immunoglobulin, an integrin, a receptor, a receptor ligand, an aptamer, a small molecule, or a variant thereof. Any number of cell surface proteins may also be included in a micro-vesicle to facilitate the binding of the micro-vesicle to a target cell and/or to facilitate the uptake of the micro-vesicle into a target cell. Integrins, receptor tyrosine kinases, G-protein coupled receptors, and membrane-bound immunoglobulins suitable for use with embodiments of this invention will be apparent to those of skill in the art and the invention is not limited in this respect. Some aspects of this invention relate to the recognition that micro-vesicles are taken up by target cells, and micro-vesicle uptake results in the release of the contents of the micro-vesicle into the cytoplasm of the target cells. Using any of the cargo RNAs, described herein, or any of the therapeutic RNAs known in the art, expression of one or more genes in a target cell may be modulated, e.g., reduce or eliminated.

In some embodiments, the micro-vesicles comprising any of the fusion proteins, any of the binding RNAs, any of the cargo RNAs, and/or any of the binding RNAs fused to any of the cargo RNAs, described herein, further include a detectable label. Such micro-vesicles allow for the labeling of a target cell without genetic manipulation. Detectable labels suitable for direct delivery to target cells are known in the art, and include, but are not limited to, fluorescent proteins, fluorescent dyes, membrane-bound dyes, and enzymes, for example, membrane-bound or cytosolic enzymes, catalyzing the reaction resulting in a detectable reaction product. Detectable labels suitable according to some aspects of this invention further include membrane-bound antigens, for example, membrane-bound ligands that can be detected with commonly available antibodies or antigen binding agents.

### Methods

Aspects of the invention include methods of delivering one or more cargo RNA precursors (i.e., cargo RNA prodrugs), such as viral targeting siRNA precursors (i.e., RNAi prodrug), for example, a precursor siRNA cargo RNA associated with a binding RNA (e.g., a shRNA having a sequence that binds to a sequence of a viral target RNA associated with a TAR element), to a target cell. In some embodiments, the cargo RNA is loaded into a micro-vesicle by co-expressing in a cell the cargo RNA associated with a binding RNA (e.g., a TAR element) and a TSG101 associating protein fused to an RNA binding protein (e.g., a Tat protein). The target cell can be contacted with a micro-vesicle in different ways. For example, a target cell may be contacted directly with a micro-vesicle as described herein, or with an isolated micro-vesicle from a micro-vesicle producing cell. The contacting can be done in vitro by administering the micro-vesicle to the target cell in a culture dish, or in vivo by administering the micro-vesicle to a subject (e.g., parenterally or non- parenterally).

It should be appreciated that the target cell may be of any origin, for example from an organism. In some embodiments, the target cell is a mammalian cell. Some non-limiting examples of a mammalian cell include, without limitation, a mouse cell, a rat cell, hamster cell, a rodent cell, and a nonhuman primate cell. In some embodiments, the target cell is a human cell. It should also be appreciated that the target cell may be of any cell type. For example, the target cell may be a stem cell, which may include embryonic stem cells, induced pluripotent stem cells (iPS cells), fetal stem cells, cord blood stem cells, or adult stem cells (i.e., tissue specific stem cells). In other cases, the target cell may be any differentiated cell type found in a subject. In some embodiments, the target cell is a cell in vitro, and the method includes administering the micro-vesicle to the cell in vitro, or co-culturing the target cell with the micro-vesicle-producing cell in vitro. In some embodiments, the target cell is a cell in a subject, and the method comprises administering the micro-vesicle or the micro-vesicle-producing cell to the subject. In some embodiments, the subject is a mammalian subject, for example, a rodent, a mouse, a rat, a hamster, or a non-human primate. In some embodiments, the subject is a human subject.

### Delivery of Pulmonary Compositions

Aspects of embodiments of the methods include contacting a cell with a pulmonary composition that configured to deliver the composition via the respiratory tract, e.g., as described below, in a manner sufficient to deliver the pulmonary agent to the cell. The cell being contacted with a pulmonary agent can be any suitable cell, where cells of interest include cells of the respiratory system. According to some embodiments, the cell is an epithelial cell, where in some instances the cell is a pulmonary cell. In some embodiments, the cell is a mammalian cell. In certain embodiments, the mammalian cell is a human cell. According to some embodiments, the cell is in vitro. In some embodiments, the cell is in vivo. Contact of the delivery composition with the cell may be achieved using any convenient protocol, where the particular protocol employed may depend on the environment of the target cell. For example, where the cell is in vitro, contact may be achieved by introducing the composition into the media of the cell, by introducing the cell into the composition, etc. Where the cell is in vivo, contact may be achieved by administering the composition to a subject harboring the cell, where the administration protocol may be local or systemic, as desired.

Aspects of embodiments of the methods include methods of treating or preventing a lung condition, e.g., COVID-19, in a subject by administering to a subject in need thereof an effective amount of a micro-vesicle containing pulmonary composition as described herein. By “an effective amount” is meant the concentration of the micro-vesicle active agent that is sufficient to elicit the desired biological effect (e.g., treatment or prevention of the lung condition). By “treatment” is meant that at least an amelioration of the symptoms associated with the condition afflicting the host is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely inhibited, e.g., prevented from happening, or stopped, e.g., terminated, such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease (e.g., infection); and/or (iii) relief, that is, causing the regression of clinical symptoms. In the context of SARS-CoV-2 virus infection, the term “treating” includes any or all of: reducing the number of viral-infected cells in patient samples, inhibiting viral replication in the cells, and ameliorating one or more symptoms associated with an infection. By “prevention” is meant that the subject at risk of acquiring a respiratory condition is not infected despite exposure to the microorganism under conditions that would normally lead to the lung condition. In some cases, the administering of the subject pulmonary composition protects the subject against infection instantaneously, for 1 day or more, 3 days or more, 1 week or more, 2 weeks or more, 3 weeks or more, 1 month or more, 2 months or more, 3 months or more, etc.

In certain embodiments of the subject methods, the composition is administered by inhalation, e.g., intranasally. Any suitable means of intranasal delivery can be used. In certain embodiments, the composition is administered intranasally in an aerosol. The composition can be administered intranasally using any device disclosed herein, including but not limited to, an inhaler, an atomizer, a nebulizer or a ventilating device. Ventilating devices include, but are not limited to, a non-invasive positive pressure ventilating device and a mechanical ventilating device. Non-limiting examples of non-invasive positive pressure ventilating device include a CPAP (Continuous Airway Pressure) machine and a BPAP (Bilevel Positive Airway Pressure) machine.

The amount of the subject composition administered can be determined using any convenient methods to be an amount sufficient to produce the desired effect in association with a pharmaceutically acceptable diluent, carrier or vehicle. The specifications for the unit dosage forms of the present disclosure will depend on the particular composition employed and the effect to be achieved, and the pharmacodynamics associated with each composition in the host. Single or multiple doses of the subject compositions can be administered according to the subject methods to provide for protection of the subject form infection for an extended period of time. In some embodiments, a single dose of the subject composition is administered. In other embodiments, multiple doses of the subject composition are administered. In certain embodiments the subject is administered at least one, two, three, four, five, six, seven, eight, nine, or ten doses of the compositions disclosed herein. The timing and dosage amounts can be readily determined using conventional methods. The subject methods may comprises administering according to a dosing schedule. Where multiple doses are administered over a period of time, the subject compound can be administered twice daily (qid), daily (qd), every other day (qod), every third day, three times per week (tiw), or twice per week (biw) over a period of time. For example, a composition can be administered qid, qd, qod, tiw, or biw over a period of from one day to about 2 years or more. For example, a composition can be administered at any of the aforementioned frequencies for one week, two weeks, one month, two months, six months, one year, or two years, or more, depending on various factors. Single or multiple doses of the subject compositions can be administered according to the subject methods at any suitable period of time before or after exposure to the microbe causing the lung condition. In certain embodiments, the subject compositions are administered one day or more, two days or more, three days or more, four days or more, five days or more, six days or more, a week or more, two weeks or more, three weeks or more, a month or more, two months or more, three months or more, four months or more, five months or more, six months or more, a year or more or two years or more prior to the exposure to the microbe causing the lung condition. According to some embodiments, the subject compositions are administered one day or more, two days or more, three days or more, four days or more, five days or more, six days or more, a week or more, two weeks or more, three weeks or more, a month or more, two months or more, three months or more, four months or more, five months or more, six months or more, a year or more or two years or more after the exposure to the microbe causing the lung condition.

### Additional Method Steps

In some embodiments, the subject methods include a step of determining or diagnosing whether the subject has a disease condition, e.g., COVID-19. The determining step can be performed using any convenient methods. In some cases, the determining step includes obtaining a biological sample from the subject, such as a nasal sample, and assaying the sample for the presence of viral nucleic acids, e.g., via RT-PCR, isothermal amplification, etc. In some cases, the determining step includes obtaining a biological sample from the subject, such as blood sample, and assaying the sample for the presence of antibodies to the virus.

Any of a variety of methods can be used to determine whether a treatment method is effective. For example, a biological sample obtained from an individual who has been treated with a subject method can be assayed for the presence and/or level of cells infected with the virus. Assessment of the effectiveness of the methods of treatment on the subject can include assessment of the subject before, during and/or after treatment, using any convenient methods. Aspects of the subject methods further include a step of assessing the therapeutic response of the subject to the treatment.

In some embodiments, the method includes assessing the condition of the subject, including diagnosing or assessing one or more symptoms of the subject which are associated with the disease or condition of interest being treated (e.g., as described herein). In some embodiments, the method includes obtaining a biological sample from the subject and assaying the sample, e.g., for the presence of virus or components thereof that are associated with the disease or condition of interest (e.g., as described herein). The assessment step(s) of the subject method can be performed at one or more times before, during and/or after administration of the subject compounds, using any convenient methods. In certain cases, the assessment step includes identification and/or quantitation of viral cells. In certain instances, assessing the subject include diagnosing whether the subject has a lung condition or symptoms thereof.

In some embodiments the method includes determining whether a subject has been exposed to a pathogenic virus, such as a coronavirus, e.g., SARS-CoV-2. Any convenient determination method may be employed, such as a contact tracing method. Such methods may be employed where one wishes to prevent a subject from suffering from a disease condition, such as COVID-19, e.g., where the methods are use in a prophylactic manner to prevent a subject from suffering from a disease condition, e.g., following exposure to a pathogenic virus.

A variety of subjects may be amenable to treatment using the subject methods and compositions disclosed herein. As used herein, the terms “subject” and “host” are used interchangeably. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). According to some embodiments, the subject is a human. As such, in some cases, the subject is one who has a lung condition. In certain cases, the subject is one who is at risk of having or is suspected of having a lung condition.

### Pharmaceutical Compositions

Other aspects of the present disclosure relate to pharmaceutical compositions comprising any of the micro-vesicles or micro-vesicle (e.g., ARMM) producing cells provided herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (24) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for delivering a cargo RNA (e.g., a cargo RNA is an shRNA having a sequence that binds to target viral RNA) to a cell. Suitable routes of administrating the pharmaceutical composition described herein include, without limitation: pulmonary, topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition is a pulmonary composition configured to be delivered to the respiratory tract of a subject in need thereof. In addition to the micro-vesicles, pulmonary agent delivery compositions employed in embodiments of the methods may include one or more additional components. In certain embodiments, the pulmonary agent delivery compositions may further include one or more additional components. Any convenient excipients, carriers, or other components, etc. can be utilized in the compositions. Pharmaceutically acceptable carriers that find use in the compositions may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer’s dextrose, dextrose and sodium chloride, lactated Ringer’s or fixed oils. Intravenous vehicles include fluid and nutrient replenishers, electrolyte replenishers (such as those based on Ringer’s dextrose), and the like. Preservatives and other additives may also be present such as, for example, antimicrobials, antioxidants, chelating agents, and inert gases and the like. Examples of carriers which may be used include, but are not limited to, alum, microparticles, liposomes, and nanoparticles. Any convenient additives can be included in the subject compositions to enhance the delivery of the subject pulmonary agent. Additives of interest include, cellular uptake enhancers, carrier proteins, lipids, dendrimer carriers, carbohydrates, and the like. When present, these one or more additional components, collectively referred to as the vehicle, may make up any desired amount of the delivery composition.

The pulmonary composition may be present in any convenient format, such as in liquid format, dry format, etc. In some instances, the composition is a lyophilized composition. Lyophilization, also known as freeze-drying or cryodesiccation, is a low temperature dehydration process that involves freezing the product, lowering pressure, then removing the ice by sublimation. This process is in contrast to dehydration by most conventional methods that evaporate water using heat. Where the composition is a lyophilized composition, the composition may be reconstituted, e.g., by combination with a suitable amount of a liquid, such as described above, e.g., an aqueous liquid, prior to use, e.g., contact with the cell. With respect to freeze dried compositions, embodiments of the invention include freeze dried compositions that include a pulmonary agent and a transfection agent, but not a deproteinized pollen shell component.

In some embodiments of the subject methods, the pulmonary composition is an inhalable composition. In certain embodiments of the subject methods, the composition is aerosolized. In some embodiments, the aerosol comprises particles having an average particle size of 1 to 100 micrometers in diameter. According to certain embodiments, the aerosol comprises particles having an average particle size of 1 to 1.5, 1.5 to 2, 2 to 2.5, 2.5 to 3, 3 to 3.5, 3.5 to 4, 4 to 4.5, 4.5 to 5, 5 to 5.5, or 5.5 to 6 micrometers in diameter.

The pulmonary agent delivery compositions can be made using any convenient protocol and aspects of the invention further include methods of making the pulmonary agent delivery compositions disclosed herein. The fabrication methods may include combining the pulmonary agent and the deproteinized pollen shell component to produce the pulmonary agent delivery composition. In some embodiments, the methods include combining the pulmonary agent, the deproteinized pollen shell component and the transfection agent. In certain embodiments, the methods include combining the pulmonary agent and the transfection agent. The methods may further include aerosolizing the composition. In some embodiments, the methods include lyophilizing the compositions. In certain embodiments, the methods include lyophilizing and reconstituting the compositions.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer’s or Hank’s solution. In addition, the pharmaceutical composition can be in solid forms and re-dissolved or suspended immediately prior to use. Lyophilized forms are also contemplated.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

### Kits

Aspects of the present disclosure also include kits. Some aspects of this disclosure provide kits comprising a nucleic acid construct comprising a nucleotide sequence encoding one or more of any of the proteins (e.g., TSG101 associating protein, and TSG101), fusion proteins (e.g., ARRDC1-Tat), and/or RNAs (e.g., TAR, TAR-cargo RNA) provided herein. In some embodiments, the nucleotide sequence encodes any of the proteins, fusion proteins, and/or RNAs provided herein. In some embodiments, the nucleotide sequence comprises a heterologous promoter that drives expression of any of the proteins, fusion proteins, and/or RNAs provided herein.

In some embodiments, the kit comprises an expression construct encoding a binding RNA (e.g., TAR) fused to a cargo RNA as described herein. In some embodiments, a further encodes a binding RNA (e.g., TAR) and/or a cargo RNA. Some aspects of this disclosure provide kits comprising a nucleic acid construct, comprising (a) a nucleotide sequence encoding a TSG101 protein fused to an RNA binding protein (e.g., Tat) as provided herein; and (b) a heterologous promoter that drives expression of the sequence of (a).

Some aspects of this disclosure provide micro-vesicle (e.g., ARMM) producing cells comprising any of the proteins, fusion proteins, and/or RNAs provided herein. In some embodiments, the cells comprise a nucleotide that encodes any of the proteins, fusion proteins, and/or RNAs provided herein. In some embodiments, the cells comprise any of the nucleotides or vectors provided herein.

A pharmaceutical composition can be provided as a pharmaceutical kit comprising (a) a container containing a micro-vesicle or micro-vesicle producing cell of the invention and (b) a second container containing a pharmaceutically acceptable diluent (e.g., sterile water) for administration. The pharmaceutically acceptable diluent can be used e.g., for reconstitution or dilution of the micro-vesicle or micro-vesicle producing cell of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer’s solution, or dextrose solution. It may further include other materials desirable from a commercial and user standpoint, including other buffers, diluents, filters, needles, syringes, and package inserts with instructions for use.

Components of the kits may be present in separate containers, or multiple components may be present in a single container. In addition to the above-mentioned components, a subject kit may further include instructions for using the components of the kit, e.g., to practice the subject methods. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Utility

Micro-vesicle compositions as described herein find use in a variety of applications, including but not limited to therapeutic applications. Among other applications, micro-vesicles in accordance with embodiments of the invention may be used to treat subjects for a variety of different conditions, such as disease conditions. The disease conditions may be modulated by a target RNA(s) for which the cargo prodrug RNA is configured to target, where the target RNA may vary. In some instances, the target RNA is an RNA endogenous to the target cell (and in some instances also the micro-vesicle producing cell). In other instances, the target RNA is exogenous to the target cells (and instances also the micro-vesicle producing cell), e.g., where the target RNA is pathogenic organism target RNA, such as pathogenic viral RNA, e.g., as present in pathogenic viral disease conditions, such as but not limited to, coronaviral mediated pathogenic disease conditions, such as COVID-19, SARS, MERS, etc., influenza mediated pathogenic disease conditions, and the like. Additional types of diseases, disorders and/or conditions that may be treated using micro-vesicles of the invention, e.g., as described herein, include, but are not limited to, diseases, disorders, and/or conditions characterized by dysfunctional or aberrant protein or polypeptide activity and include, but are not limited to, rare diseases, infectious diseases (as both vaccines and therapeutics), cancer and proliferative diseases, genetic diseases, autoimmune diseases, diabetes, neurodegenerative diseases, cardio- and reno-vascular diseases, and metabolic diseases.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### 1. Micro-Vesicle Mediated RNA-Silencing Therapy for Coronavirus Diseases

### A. Introduction

The following discloses a therapeutic entity capable of highly specific prevention of coronavirus replicase synthesis at the entry point for virus infection-which may be epithelial cells lining the nasopharynx-and also may be present in subsequently infected epithelial cells that line the lower respiratory tract. The therapeutic includes gene-silencing small RNAs that target multiple sites of the virus RdRP (RNA-dependent RNA polymerase) gene, which is essential for virus reproduction, where the small RNAs can be packaged as prodrugs into, and can be delivered therapeutically by, a unique type of micro-vesicle called ARMMS (Nabhan et al., PNAS (2012) 109 (11) 4146-4151).

Coronaviruses have been known to infect humans for more than five decades and prior to the advent of COVID-19, at least two other Coronaviruses (SARS-CoV and MERS-CoV) have caused major epidemics. The prospect that other yet-unidentified coronaviruses may emerge as significant human pathogens-perhaps by transmission from animal reservoirs-is an ongoing world health concern.

Like other viruses that infect humans through the respiratory tract, SARS-CoV-2-the cause of COVID-19-enters the human body by infecting cells of the nasopharynx, where it reproduces; its progeny then move on to, and attack, cells lower in the respiratory tract. Once inside a cell, the coronavirus RNA genome acts as a messenger RNA (mRNA) that is translated by the infected cell’s ribosomes to produce viral proteins. A segment of one of these proteins is the virus’s “replicase”, which generates multiple copies of the viral genome. Without RdRP function, the virus cannot reproduce. Coronavirus RdRP has been validated as a major molecular target of systemically administered antiviral drugs.

Cytokine release and an inflammatory response in lower respiratory tract cells result in accumulation of fluid and debris in alveoli of lungs, interfering with the transport of oxygen and carbon dioxide to and from adjacent capillaries and leading to respiratory failure. While COVID-19 is recognized as a systemic disease, its morbidity and mortality, in addition to its pathogenesis, result importantly from virus attack on epithelial cells that line the respiratory tract. It is these cells that are targeted directly by the therapeutic agent described herein.

RNAi (RNA interference) silences gene expression at the mRNA level using small RNA complementary to nucleotides in the mRNA it targets. The short-lived gene silencing agent (small interfering RNA; siRNA) is generated by cellular processing of a stable duplex RNA precursor by an enzyme called DICER. Targeting of specific mRNA sites can be engineered to be highly selective. While clinical applications of RNAi-based therapies administered locally or by intravenous injection currently exist, delivery of gene- silencing RNAs to their targets in a form that is undegraded by either extracellular enzymes or endocytosis-mediated mechanisms has been particularly challenging. A further factor that has limited the development of RNAi for use in antiviral therapy has been the ability of viruses to mutate and evade the sequence-specific targeting properties of the therapy. The therapeutic approach described here is designed to address these issues.

During the past decade exosomes and other membrane-encapsulated extracellular vesicles derived from intracellular organelles have been explored as delivery agents for therapeutic cargo. However, attempts at exosome mediated therapeutic applications have been limited by the lack of uniformity and quantitative control of exosome production, and also by the limited ability to control the packaging of cargo into these vesicles. In 2012, a unique type of extracellular vesicle that, unlike exosomes, is formed by outward budding of the plasma membrane of cells was reported (Nabhan et al., PNAS (2012) 109 (11) 4146-4151). Direct plasma membrane budding (DPMB) was found to be dependent on the production of the arrestin-like cellular protein, ARRDC1. ARRDC1 localizes to the plasma membrane, recruiting TSG101 to the site. The event causes the membrane to bud outward at that location and form a micro-vesicle that is released into the extracellular space. These ARRDC1-Mediated Micro-vesicles formed by direct plasma membrane budding (DPMB) are known as ARMMS.

The mechanism underlying the formation of these vesicles by direct plasma membrane budding allows for the strategy employed in the present invention to package RNA and protein synthesized in ARMMS-producing cells into ARMMS. RNAs that are physically attached to ARRDC1 using a naturally occurring linkage system mediated by the HIV TAT protein and its RNA ligand, TAR, are transferred into the vesicle along with ARRDC1. U.S. Pat. describing this mechanism include 9,816,080 and 10,260,055, the disclosures of which are herein incorporated by reference.

After ARMMS are released from the producing cells, they can fuse with plasma membranes of other cells they may contact and discharge their cargo directly into the cytoplasm of those cells. This process, which has been shown to have a role in normal intercellular communication, avoids the actions of proteases and nucleases that degrade macromolecules entering cells by endocytic mechanisms. Whereas ARMMS are produced physiologically in nature, we found that their production can be controlled and increased dramatically by engineered over-expression of ARRDC1.

### B. Technical Summary

Described below is the novel use of ARMMS to deliver multiple stable shRNA precursors concurrently to virus-infected cells as prodrugs of gene-silencing siRNA. The prodrugs, i.e., also referred to herein as siRNA precursors, include shRNA sequences that target different RdRP sites. The prodrugs are synthesized concurrently and biologically in ARMMS-producing cells by DNAs inserted chromosomally in those cells. DNAs encoding the shRNAs are fused to a sequence encoding TAR RNA, enabling multiple species of shRNA molecules that attack different sites of the target to be attached to a population of ARRDC1-TAT fusion molecules produced by these cells, which enables the shRNAs to be distributed into the ARMMS released from the cells.

Target sequences for shRNAs have been computationally identified that correspond to regions of RdRP that are 1) common to all coronaviruses known to infect humans, and 2) have not been mutated in COVID-19 isolates for which the RdRP sequence is publicly available. These conditions suggest that sequence conservation at these loci is required for the function of the viral replicase. Sequences corresponding to different sites identified computationally and also determined to have structural features conducive for RNAi were synthesized chemically and tested for ability to produce siRNA that reduces production of RdRP from a construct that was introduced human 293T cells by co-transfection; two of these sequences are: shRDRP-1:

AGGAAGTTCTGTTGAATTAAA (SEQ ID NO:9)

and shRDRP-8:

CTGCATTGTGCAAACTTTAAT (SEQ ID NO:10)

. The above sequences are delivered by ARMMS to A549 human lung epithelial cells expressing RdRP RNA from a chemically-synthesized sequence that we have inserted chromosomally.

Utility of the ARMMS-mediated gene-silencing strategy described here requires that the cell population producing shRNA-TAR fusion molecules from chromosomally encoded sites, and also producing ARMMS, be devoid of the endoribonuclease, DICER-which initiates processing of duplex shRNA into short 21-23 nt functionally active RNA fragments. If DICER is present in the ARMMS-producing cells, cleavage of the shRNA will break the shRNA-TAR link that enables the shRNA to be attached to ARRDC1-TAT-precluding loading of the shRNA prodrug into ARMMS. Such processing into active gene-silencing siRNA would also lead to degradation of the gene silencing agent.

To ensure that RNA synthesized in ARMMS-producing cells remains as a stable inactive prodrug we will generate ARMMS from a 293T human cell line containing inactivating mutations in all three DICER genes (Bogerd et al., RNA (2014) 20:923-937) to express the shRNA. The production of both prodrug and ARMMS in such cells assures that the prodrug shRNA remains unprocessed, and is attached to TAR (and thus, to ARRDC1-TAT) during ARMMS production. The resultant AARMS are expected to be deficient in DICER activity, in contrast to AARMS produced by cells that do not include inactivity mutations in DICER genes. After ARMMS-mediated delivery of prodrug shRNAs to epithelial cells, which lack DICER mutations, the shRNA prodrug is separated from the TAT- TAR linkage and processed into active siRNA fragments. The presence and quantity of each prodrug shRNA sequence in ARMMS isolated and purified as previously described is determined by RT-PCR using primers complementary to the sequences of each of the shRNA-TAR constructs. Amounts of each small-RNA prodrug made in ARMMS- producing cells and delivered to targeted cells is quantified by RT-PCR using primers complementary to the different sequences. Efficiency of delivery of cargo by ARMMS delivered intra-nasally into the respiratory tract by inhalation is tested using ARRDC-1 that has been tagged by fusion with Green Fluorescence Protein (GFP).

Together, the results obtained will show the validity of the therapeutic approach described here. The ability to control micro-vesicle production, to generate stable prodrug cargo in cells that produce ARMMS, and to deliver a heterogeneous prodrug population that is converted to active siRNA in targeted cells are key technical features of the work described herein.

This work provides a prophylactic and therapeutic strategy that enables interfering RNA to be used effectively to combat coronavirus, as well as other respiratory viral infections.

### II. Constructs of TAR-myc-shSupt4 and TAR-myc-shRdRP Are Efficient to Knock Down Target Gene RNA Abundance

### A. Experimental Procedure

1. 10⁶ 293 dicer-deficient-RdRP cells were plated in 1 well of 6 well plates and transfected with 1 µg TAR-myc-shRNA constructs.

2. Cells transfected with TAR-myc-shSupt4 were collected after 48h of growth. Cells transfected with TAR-myc-shRdRP were collected after 48h of growth, then 7d with 1 µg/ml puromycin selection (the “myc” domain in these constructs and those described below functions only as a linker domain and was arbitrarily chosen).

3. Cellular RNA was extracted with RLT lysis buffer and purified with RNAeasy mini kit (Qiagen).

4. Reverse transcription was performed with iScript RT mix (Bio-Rad), and Taqman real time PCR was used for the quantification of cellular supt4 / rdRp RNA abundance. The relative fold change of transcript abundance normalized with hGAPDH was shown in Y-axis.

### B. Results

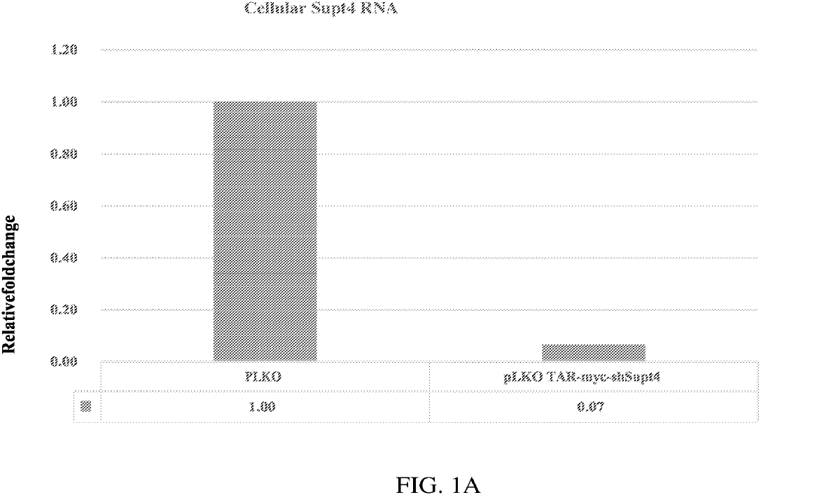

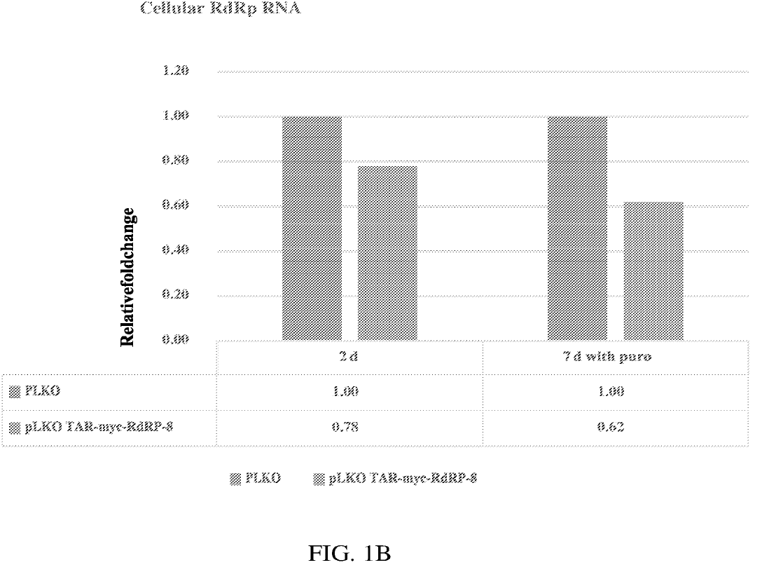

FIG. 1A shows that 2 day TAR-myc-shSupt4 transfection decreases cellular supt4 RNA abundance. FIG. 1B shows 2 day and 7 day TAR-myc-shRdRP transfection decreases cellular RdRP RNA abundance.

### III. Interaction of TAT-TAR Enables ARRDC1-mediated shRNA Packaging in ARMMs

### A. Experimental Procedure

1. 5X10⁶ 293 dicer deficient cells were plated in 10 cm plate and transfected with 2.5 µg HA-ARRDC1-TAT & 2.5 µg TAR-myc-shRdRP, 2.5 µg HA-ARRDC1 & 2.5 µg TAR-myc-shRdRP, 2.5 µg HA-ARRDC1-TAT & 2.5 µg TAR-myc-shSupt4, 2.5 µg HA-ARRDC1 & 2.5 µg TAR-myc-shSupt4 respectively.

2. After 24h of culture, culture medium was removed. Cells were washed 2 times with Wash PBS and split into 3 10 cm plates to grow for additional 48 hours.

3. The media were collected for ARMMs purification. The cells were collected for protein and RNA extraction.

4. RNA from purified ARMMs was extracted using miRNAeasy serum/plasma kit (Qiagen) plus on column Dnase I digestion. The eluted RNA was reverse transcribed with MMLV (Thermo Fisher), and Taqman real time PCR was used for the quantification of shRNA abundance in ARMMs. The relative fold change of transcript abundance normalized with spike-in control tomato GAPDH was shown in Y-axis.

5. Protein from purified ARMMs was extracted by adding lysis buffer and boiling in SDS loading buffer for 10 min at 70ºC. ARMMs protein was separated on 5-12% SDS gel, blotted to PVDF membrane, and detected with HA antibody for ARRDC1 protein abundance.

### B. Results

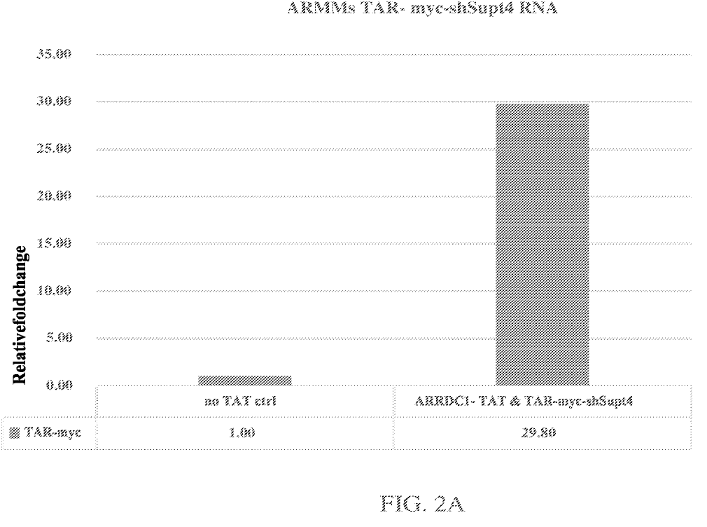

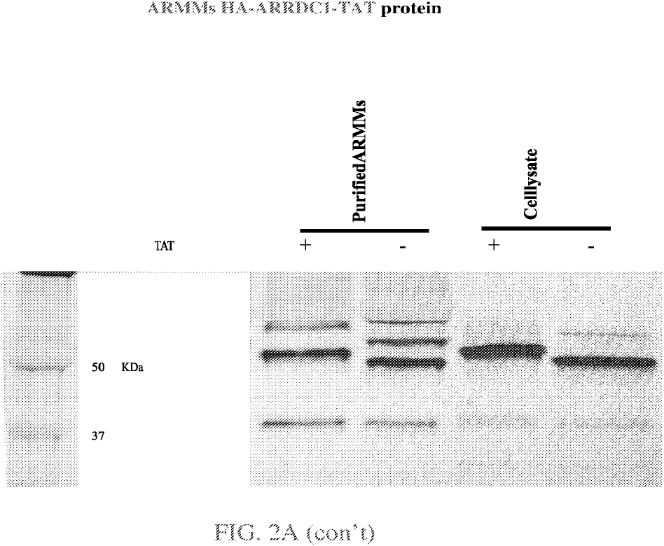

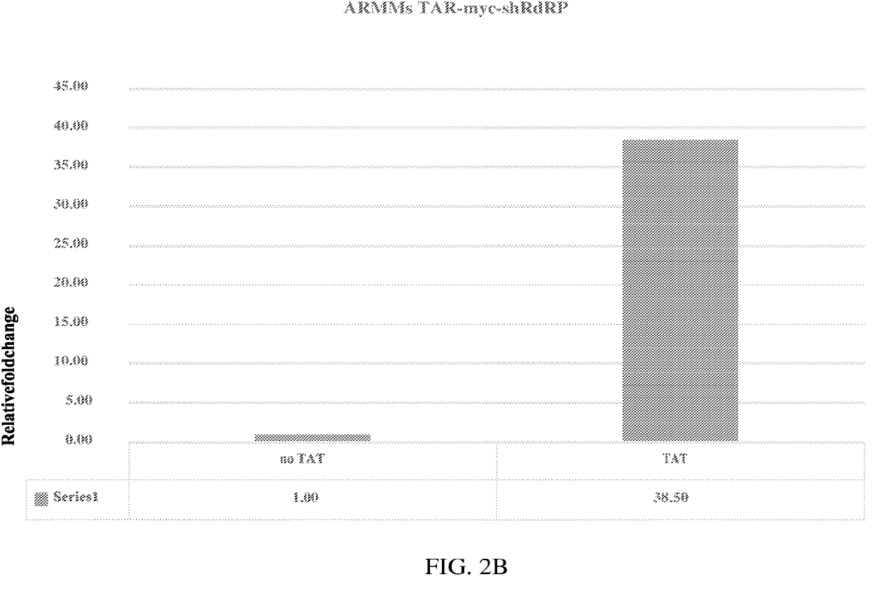

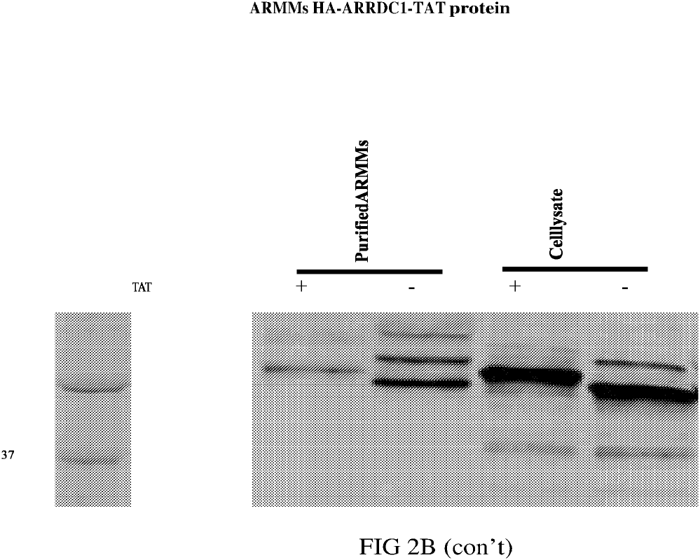

FIG. 2A shows higher TAR-myc-shSupt4 content in ARMMs prepared from 293 dicer deficient cells transfected with ARRDC1-TAT as compared with ARRDC1 (no TAT). FIG. 2B shows higher TAR-myc-shRdRP content in ARMMs prepared from 293 dicer deficient cells transfected with ARRDC1-TAT as compared with ARRDC1 (no TAT).

### IV. Elevated Amounts of shRNA Is Present in ARMMs Produced From 293 Dicer deficient cells as compared with normal 293 cells

### A. Experimental Procedure

1. 5×10⁶ 293-dicer deficient cells or 293 cells were plated in 10 cm plate, and transfected with 2.5 µg HA-ARRDC1-TAT & 2.5 µg TAR-myc-shRdRP, 2.5 µg HA-ARRDC1-TAT & 2.5 µg TAR-myc-shSupt4 respectively.

2. After 24h of culture, culture medium was removed. Cells were washed 2 times with Wash PBS, and split into 3 10 cm plate to grow for additional 48 hours.

3. The medium was collected for ARMMs purification. Cells were collected for protein and RNA extraction.

4. RNA from purified ARMMs was extracted using miRNAeasy serum/plasma kit (Qiagen) plus on column Dnase I digestion. The eluted RNA was reverse transcribed with MMLV (Thermo Fisher), and Taqman real time PCR was used for the quantification of shRNA abundance in ARMMs.

5. Protein was extracted from purified ARMMs by adding lysis buffer and boiling in SDS loading buffer for 10 min at 70C. ARMMs protein was separated on 5-12% SDS gel, blotted to PVDF membrane, and detected with HA antibody for ARRDC1 protein abundance.

6. Cellular RNAs were extracted with RLT lysis buffer and purified with RNAeasy mini kit (Qiagen). Reverse transcription was performed with iScript RT mix (Bio-Rad), and Taqman real time PCR was used for the quantification of cellular supt4 / rdRp RNA abundance. The relative fold change of transcript abundance normalized with hGAPDH was shown in Y-axis.

### B. Results

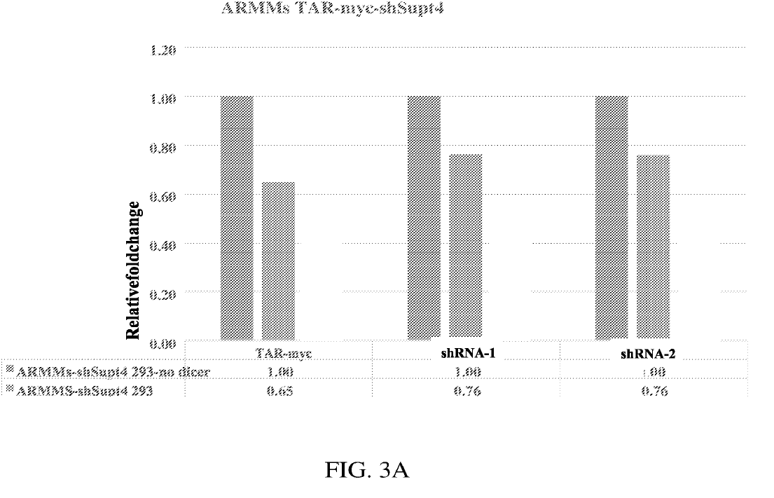

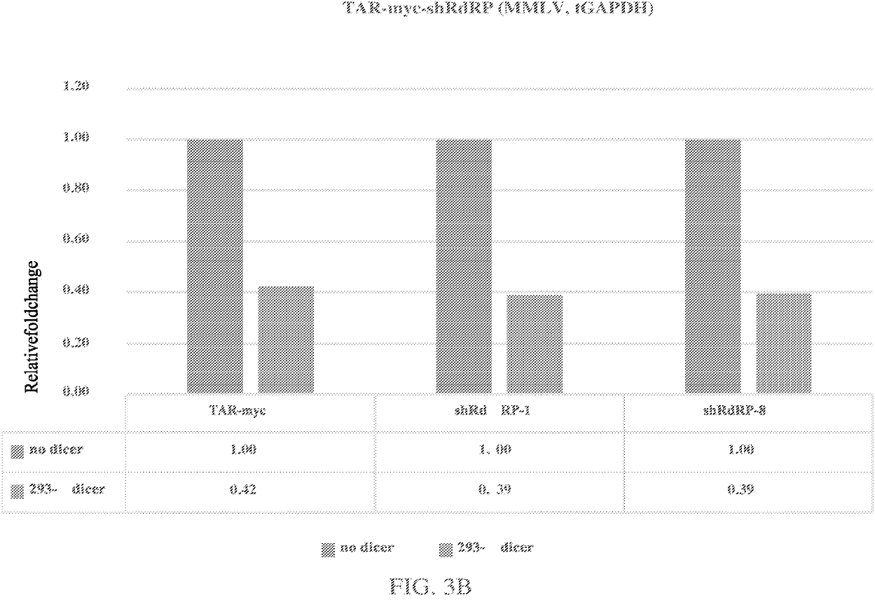

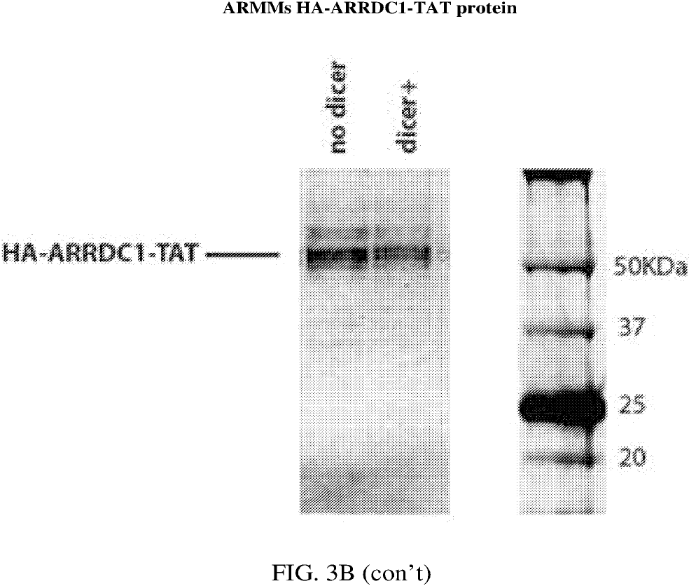

FIG. 3A shows higher TAR-myc-shSupt4 content in ARMMs prepared from 293-dicer deficient cells (labeled 293-no dicer) as compared with ARMMs from 293. FIG. 3B shows higher TAR-myc-shRdRP content in ARMMs prepared from 293 dicer deficient cells as compared with ARMMs from 293.

### V. Protection of ARMMs Producing Cells From the Toxicity Effect of Supt4 siRNA by Using 293-no-dicer Cells

### A. Experimental Procedure

1. 5×10⁶ 293 dicer deficient or 293 cells were plated in 10 cm plate, and transfected with 2.5 µg HA-ARRDC1-TAT & 2.5 µg TAR-myc-shSupt4 respectively.

2. After 24h of culture, culture medium was removed. Cells were washed 2 times with Wash PBS, and split into 3 10 cm plate to grow for additional 48 hours.

3. Medium was collected for ARMMs purification. Cells were collected for protein and RNA extraction.

4. RNA was extracted from purified ARMMs using miRNAeasy serum/plasma kit (Qiagen) plus on column Dnase I digestion. The eluted RNA was reverse transcribed with MMLV (Thermo Fisher), and Taqman real time PCR was used for the quantification of shRNA abundance in ARMMs.

5. Protein was extracted from purified ARMMs by adding lysis buffer and boiling in SDS loading buffer for 10 min at 70ºC. ARMMs protein was separated on 5-12% SDS gel, blotted to PVDF membrane, and detected with HA antibody for ARRDC1 protein abundance.

6. Cellular RNAs were extracted with RLT lysis buffer and purified with RNAeasy mini kit (Qiagen). Reverse transcription was performed with iScript RT mix (Bio-Rad), and Taqman real time PCR was used for the quantification of cellular supt4/rdRp RNA abundance. The relative fold change of transcript abundance normalized with hGAPDH was shown in Y-axis.

### B. Results

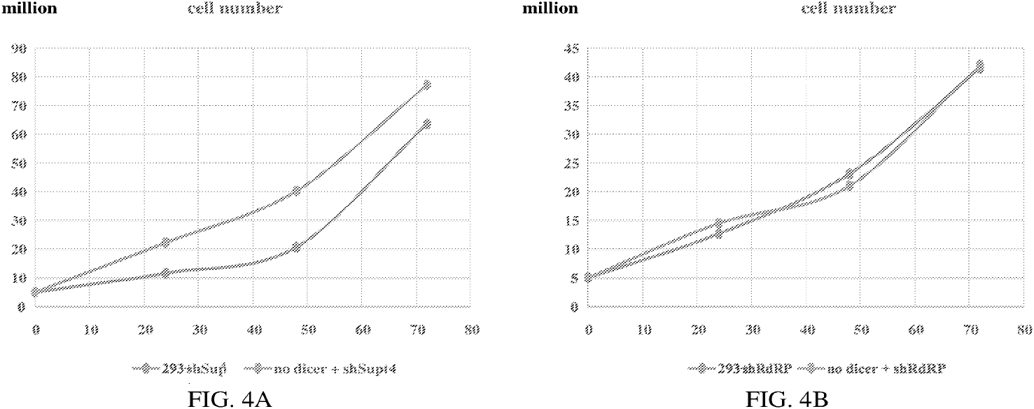

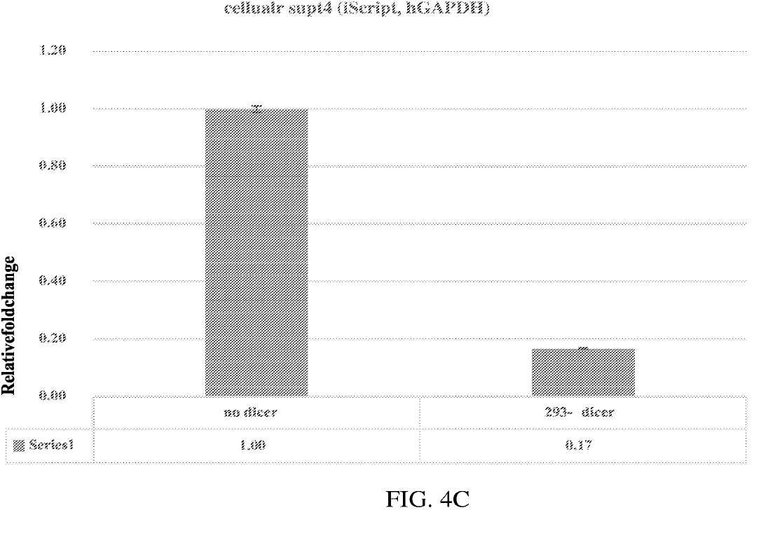

Supt4 siRNA processed by Dicer from TAR-myc-shSupt4 promotes cell apoptosis in high concentration. FIG. 4A: ARMMs-shSupt4: 293-no-dicer producing cells show higher viability compared with 293 producing cells. FIG. 4B: ARMMs-shRdRP 293-no-dicer producing cells show similar viability as 293 producing cells. FIG. 4C: cellular supt4 RNA is down-regulated in 293 ARMMs producing cells compared with 293-no-dicer ARMMs producing cells.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. §112(f) or 35 U.S.C. §112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. §112(6) is not invoked.

What is claimed is:

1-22. (canceled)

23. A method of treating a subject for a condition mediated by a target RNA, the method comprising:
administering to the subject an effective amount of a micro-vesicle comprising:
a) a TSG101 associating protein capable of associating non-covalently with TSG101;
b) an RNA-binding protein attached covalently to the TSG101 associating protein; and
c) a cargo RNA complex comprising:
(i) a binding RNA bound non-covalently to the RNA-binding protein; and
ii) a cargo prodrug RNA component;
to treat the subject for the condition.

24. The method according to claim 23, wherein the micro-vesicle comprises a plurality of distinct cargo RNA complexes each containing different cargo prodrug RNA components that target different sequences of the RNA target.

25. The method according to claim 24, wherein the micro-vesicle comprises 2 to 10 distinct cargo RNA complexes each containing different cargo prodrug RNA components that target different sequences of the RNA target.

26. The method according to claim 23, wherein the TSG101 associating protein comprises an arrestin domain containing protein 1 (ARRDC1) component.

27. The method according to claim 26, wherein ARRDC1 component comprises a fusion protein comprising the RNA-binding protein.

28. The method according to claim 23, wherein the RNA-binding protein is selected from the group consisting of: a trans-activator of transcription (Tat) protein or active variant thereof; a Rev protein or active variant thereof; an MS2 phage coat protein or active variant thereof; a P22 N protein or active variant thereof; a λ N protein or active variant thereof; a φ21 protein or active variant thereof; and an HIV-1 nucleocapsid protein or active variant thereof.

29. The method according to claim 23, wherein the binding RNA comprises: a trans-activating response element (TAR) or active variant thereof; a Rev response element (RRE) or active variant thereof; an MS2 RNA sequence or active variant thereof; a P22 boxB RNA sequence or active variant thereof; a λ boxB RNA sequence or active variant thereof; a φ21 boxB RNA sequence or active variant thereof; and a SL3 ψ RNA sequence or variant thereof.

30. The method according to claim 23, wherein the RNA-binding protein comprises Tat and the binding RNA comprises TAR.

31. The method according to claim 23, wherein the cargo RNA component comprises a precursor of an inhibitory ribonucleic acid.

32. The method according to claim 31, wherein the inhibitory ribonucleic acid comprises a siRNA, RNAi, shRNA or microRNA.

33. The method according to claim 31, wherein the cargo RNA component comprises a double-stranded ribonucleic acid that is 30 nt or longer and is capable, upon processing, of binding to a sequence in an RNA target.

34. The method according to claim 31, wherein the cargo RNA component comprises an RNA decoy.

35. The method according to claim 23, wherein the target RNA is a viral target RNA.

36. The method according to claim 35, wherein the viral target RNA is a viral target RNA of a pathogenic virus.

37. The method according to claim 36, wherein the pathogenic virus causes a respiratory disease.

38. The method according to claim 37, wherein the pathogenic virus is a coronavirus.

39. The method according to claim 38, wherein the coronavirus is SARS-CoV-2.

40. The method according to claim 39, wherein the viral RNA target encodes RNA-dependent RNA polymerase (RdRP).

41. The micro-vesicle according to claim 36, wherein the pathogenic virus is an influenza virus.

42. The method according to claim 23, wherein the target RNA is a non-viral target RNA.

43-87. (canceled)
